(19) United States
(12) Patent Application Publication
(10) Pub. No.: US 2021/0374938 A1
(43) Pub. Date: Dec. 2, 2021

(54) OBJECT STATE SENSING AND CERTIFICATION

(71) Applicant: Quivr Ai Corp., Seattle, WA (US)

(72) Inventors: Brian Monnin, Seattle, WA (US); John Pella, Redmond, WA (US); Quentin Dewolf, Seattle, WA (US); Nattapon Chaimanonart, Kirkland, WA (US); Sean Robinson, Burien, WA (US); Ben Rush, Seattle, WA (US); Greg Gottesman, Mercer Island, WA (US)

(21) Appl. No.: 17/227,101

(22) Filed: Apr. 9, 2021

Related U.S. Application Data
(60) Provisional application No. 62/704,758, filed on May 27, 2020.

(51) Int. Cl. G06T 7/00; G06T 7/187; G06T 7/70; G06T 7/50; G08B 6/00; G08B 21/02; A61L 2/28

(52) U.S. Cl. CPC G06T 7/0004; G06T 7/187; G06T 7/70; G06T 7/50; G08B 6/00; G06T 2207/30164; A61L 2/28; G06T 2207/20081; G06T 2207/10028; G06T 2207/20221; G08B 21/02

(57) ABSTRACT

Techniques for determining a state of an object are discussed herein. In some implementations, a state sensing device may be capable of sensing and/or helping determine a state of an object. A state of an object may include various information about a physical object, such as a position and/or location of the object, a condition of the object, how the object relates to one or more other objects, etc. This disclosure may also be directed to providing a certification of the state of the object. For instance, the state sensing device may help provide a degree of confidence in the determined state of the object.

### CROSS REFERENCE TO RELATED APPLICATIONS

This patent application claims priority filing benefit from U.S. Provisional Patent Application No. 62/704,758, filed May 27, 2020, which is hereby incorporated by reference, in its entirety.

### BACKGROUND

The health and well-being of people is of utmost concern in the days of dangerous microorganisms, such as bacteria and viruses. For example, the coronavirus pandemic of 2020 (“COVID-19”) resulted in global infections and deaths not seen in over a century. In the absence of a vaccine, the only way of defending against the deadly virus was through social distancing and diligent disinfection and/or sanitization techniques. The Centers for Disease Control and Prevention (“CDC”) reported that cleaning dirty surfaces followed by disinfection is a best practice measure for prevention of COVID-19 and other viral respiratory illnesses in households and community settings. However, while disinfecting surfaces, it can be difficult if not impossible to easily determine if the surface has been adequately disinfected.

Whether or not an object has been adequately disinfected may be viewed as a “state” of an object. A state of an object may include any of a variety of other classifications, such as whether an object is clean, broken, or present at a location, whether a product in a factory setting is assembled, etc. A wide variety of scenarios is contemplated related to a state of an object.

### BRIEF DESCRIPTION OF THE DRAWINGS

The detailed description is described with reference to the accompanying figures. In the figures, the left-most digit(s) of a reference number identifies the figure in which the reference number first appears. The same reference numbers in different figures indicate similar or identical items. In some cases, parentheticals are utilized after a reference number to distinguish like elements. Use of the reference number without the associated parenthetical is generic to the element. The systems depicted in the accompanying figures are not to scale and components within the figures may be depicted not to scale with each other.

FIG. 1 illustrates a pictorial flow diagram of a process for sensing a state of an object using a state sensing device.

FIG. 2 illustrates an example environment including a state sensing and various accessory devices, computing devices, and network devices.

FIG. 3 shows an illustrative functional block diagram of a state sensing device.

FIGS. 4A and 4B show illustrative examples of a display of information generated by state sensing device.

FIG. 5 is a front elevational view of a user wearing an example state sensing device.

FIG. 6 is a perspective view of an example state sensing device with a display.

FIG. 7 is a perspective view of an example state sensing device mounted on a wall in an environment with a user.

FIG. 8 is a perspective view of an example state sensing device mounted on a robot arm.

FIGS. 9A-9C are various perspective views of an example environment in which a state sensing device may be employed.

FIGS. 10A-10C are partial cutaway perspective views of an example manufacturing environment in which a state sensing device may be employed.

FIGS. 11A-11B are perspective views of another example environment including a state sensing device.

FIGS. 12-14 are flow diagrams of illustrative processes for helping determine a state of an object in accordance with the present concepts.

### DETAILED DESCRIPTION

This disclosure is generally directed to determining a state of an object. More particularly, this disclosure is directed to a state sensing device that may be capable of sensing and/or helping determine a state of an object. A state of an object may include various information about a physical object, such as a position and/or location of the object, a condition of the object, how the object relates to one or more other objects, etc. This disclosure may also be directed to providing a certification of the state of the object. For instance, the state sensing device may help provide a degree of confidence in the determined state of the object.

In some examples, the state of the object may refer to a condition of the object. A condition of an object may comprise a classification of some physical characteristic of an object, such as whether an object is wet/dry, clean/dirty, broken, etc. More specifically, a condition may include whether an object has been disinfected, for instance. In one example scenario, an airplane seat may be disinfected between flights, so that a newly boarded passenger is offered some protection from potential pathogens that may have been left behind by a previous seat occupant. A disinfection procedure may consist of spraying a disinfectant on the seat, tray table, etc., between flights. Alternatively or additionally, disinfection may be attempted via exposure to UV light, or via other methods.

For passenger safety and/or peace of mind, it may be beneficial to determine whether an attempt to disinfect the airplane seat was made between flights. Further, it may be helpful to know whether the disinfection attempt was likely to have been successful. For example, in the case where a disinfectant spray was used on an airplane seat, a state of the object may be indicative of whether the seat surface was wetted with the disinfectant spray. A wetted seat surface may indicate sufficient coverage by the disinfectant spray. A determination of whether the disinfection attempt was likely to have been successful may lead to a certification of disinfection. Such a certification may include a confidence level that the seat is safe for the new passenger. In some examples, a certification could also be provided in real-time to an employee performing the disinfection procedure, indicating that the employee has adequately completed the job and may move on to the next seat. The certification may be combined with metrics for various surfaces in an entire airplane, indicating a confidence level in overall disinfection procedures adopted by an airline company, for instance.

A wide variety of other scenarios are contemplated for using a state sensing device capable of helping to determine a state of an object. For example, a hotel may wish to adequately disinfect rooms between hotel guests. A hotel cleaning crew may be tasked with disinfecting a list of surfaces in each hotel room, such as a desk, chair, bedspread, sink, bathtub and/or shower, countertop, door handles, light switches, thermostat control, etc. Each of the surfaces may be pre-designated in a list of items that must be adequately disinfected before a certification is issued that the hotel room is ready for the next hotel guest. Similarly, an office building may include a conference room that may be reserved for use by different groups of people on different days. The conference room may have a variety of desks, chairs, tables, or other objects that ought to be disinfected between groups.

A state sensing device that may help determine a state of an object (e.g., airplane seat, hotel sink, manufacturing a product) that may be present in the environment that includes the object by a variety of means. In some cases, the state sensing device may be a worn or carried by a person into the environment, such as an employee of a cleaning company or crew that is disinfecting airplane cabins between flights. In this example the state sensing device may be a lightweight instrument that is relatively easy for a person to wear and/or bring along as they perform tasks. In other examples, the state sensing device may be fixed in an environment, such as wall-mounted in a conference room. The state sensing device may also be mounted in a moveable manner, such as on a robot arm in a factory, etc.

In order to determine the state of the object, the state sensing device may be able to sense various information about the object. In some examples, the state sensing device may include one or more sensors that allow the state sensing device to detect information about the object, such as a geometry of the object and/or an appearance of the object. For instance, the sensors may include one or more types of cameras that enable sensing of one or more surfaces of the object. The state sensing device may also be able to collect information related to an orientation of the object (e.g., which way an object is facing), a position of the object in local space (e.g., where in a hotel room the object is located), a position of the object in global space (e.g., what street, and in what city the hotel is located), etc. The sensors of the state sensing device may be used to collect information that helps determine whether the object has a wetted surface, whether UV light reached the object, or whether the object is associated with another object of interest. For instance, the sensors may be able to “see” or otherwise determine whether a particular part has been added onto a product that is being assembled in a factory setting.

In some examples, additional information may be used to help determine a state of an object or to help produce a certification for the state of the object. For instance, global positioning system (GPS) data may be used to help determine a location of the state sensing device and/or the object. In another case, various forms of crowd-sourced data may be used to help determine whether disinfection is sufficient. For instance, data from crowd-sourcing, a government agency, or another entity may suggest that a pathogen was present, currently or recently, in an area that includes objects to be disinfected. The presence of the pathogen may adjust the expectation for thoroughness of disinfection, in some cases. Thus, the criteria for issuing a certification that an object was disinfected may change based on various types of input.

In some examples, the state sensing device may include an electronics assembly for collecting data from the one or more sensors of the state sensing device, performing computing functions on the data, and/or sending the data to a remote computing device. The electronics assembly may include one or more components installed on a circuit board, such as a printed circuit board. The electronics assembly may include wireless capabilities to communicate with another computing device. The sensors on the state sensing device may include, but are not limited to, one or more of any of a camera and/or imaging sensor, gyroscope, GPS receiver, accelerometer, pressure sensor, temperature sensor, humidity sensor, pH sensor, microphone, speaker haptic componentry, power supply and/or energy module, etc. Further, the state sensing device may include a display screen for displaying information to a user.

The information gathered by the state sensing device, such as with the sensors of the state sensing device or data from other sources, may be combined to give an overall impression of the state of the object. Stated another way, information about the object may be fused to determine the state of the object. Simply declaring that an airplane seat was disinfected may not be particularly helpful. An airline company may find more value in knowing which airplane seat was disinfected, on which airplane, in which airport, and between which two flights. Furthermore, the airline company may be interested in knowing which disinfectant spray was used, which employee was doing the spraying, and/or the degree of coverage of the disinfectant spray on the surface of the particular airplane seat—both in terms of how wetted the seat surface was, and also the areal extant of the coverage of the seat surface. Fusion of the various information regarding the state of the object may help lead to a certification that gives the recipient confidence that the result may be believed. In some examples, the determination of the state of the object may be performed by the state sensing device. In other example, information collected by the state sensing device may be sent to a remote computing device that performs the determination of the state of the object. In still other examples, the information collected using the one or more sensors of the state sensing device may be partly processed by the state sensing device and partly processed remotely. Similarly, the collected information and/or data regarding the state of the device may be stored on the state sensing device and/or remotely.

To summarize, a state sensing device may include one or more sensors capable of collecting information regarding a state of an object in an environment. For example, the collected information may be used to help determine various aspects of the state of the object, such as geometry, appearance, orientation, position, association with another object, etc. The state sensing device may be a simple, lightweight instrument that may be worn or carried by a user. Fusion of the collected information related to the state of the object may produce a certification of the state of the object. For example, an airplane seat may be certified as having been adequately disinfected. In this example, certification of disinfection may provide feedback to a cleaning crew regarding job completion, may inform an airline company regarding adequacy of safety procedures, and/or may give confidence to subsequent airline passengers that their seat is relatively safe from potential pathogens. Therefore, certification assisted by the state sensing device may help improve safety and/or job performance in a variety of settings.

The techniques and systems described herein may be implemented in a number of ways. Although discussed in the context of disinfecting an airplane or hotel, the techniques may be implemented in any context and are not limited to the particular examples discussed herein. For example, the techniques can be implemented in a manufacturing context to verify the accuracy/completeness of manufacturing an object; in a hotel context to determine whether procedures such as setting a table or laying out objects was performed correctly; in a retail context to determine whether shelves were restocked or that products were presented according to a predetermined layout; in a construction context to determined whether a building was built to specification; and the like. Example implementations are provided below with reference to the following figures.

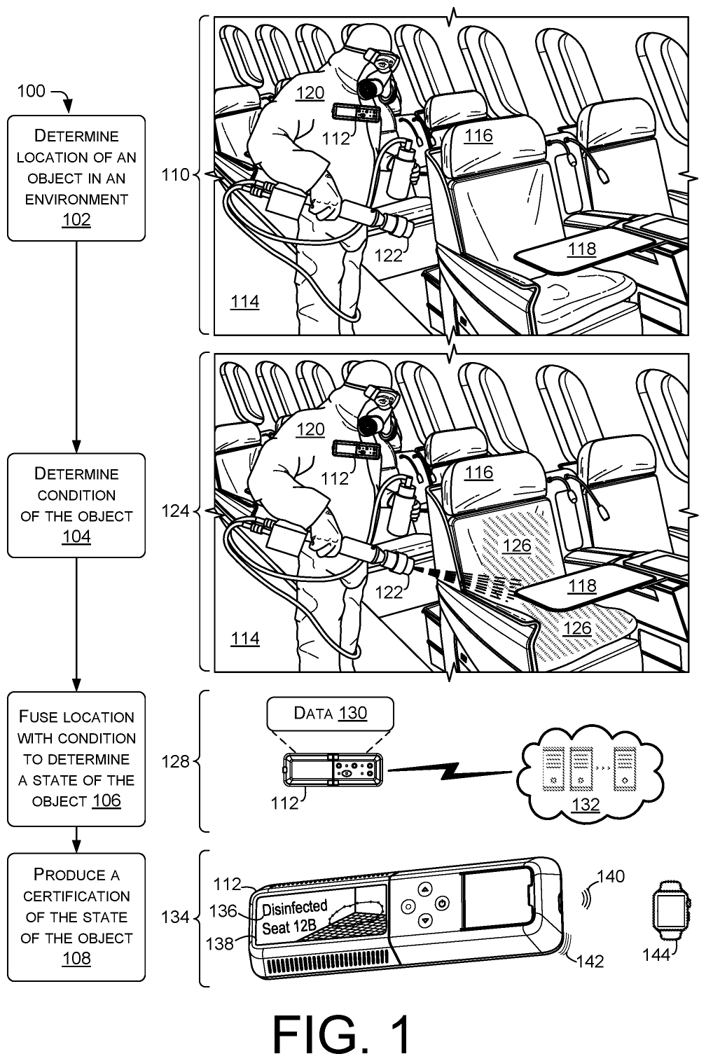

FIG. 1 illustrates a pictorial flow diagram of a process 100 for determining a state of an object. FIG. 1 illustrates a high-level pictorial flow diagram, and additional details of the implementation are given throughout this disclosure. In some examples, process 100 may include a series of operations 102-108 that generally describe a scenario in accordance with object state sensing and/or certification concepts.

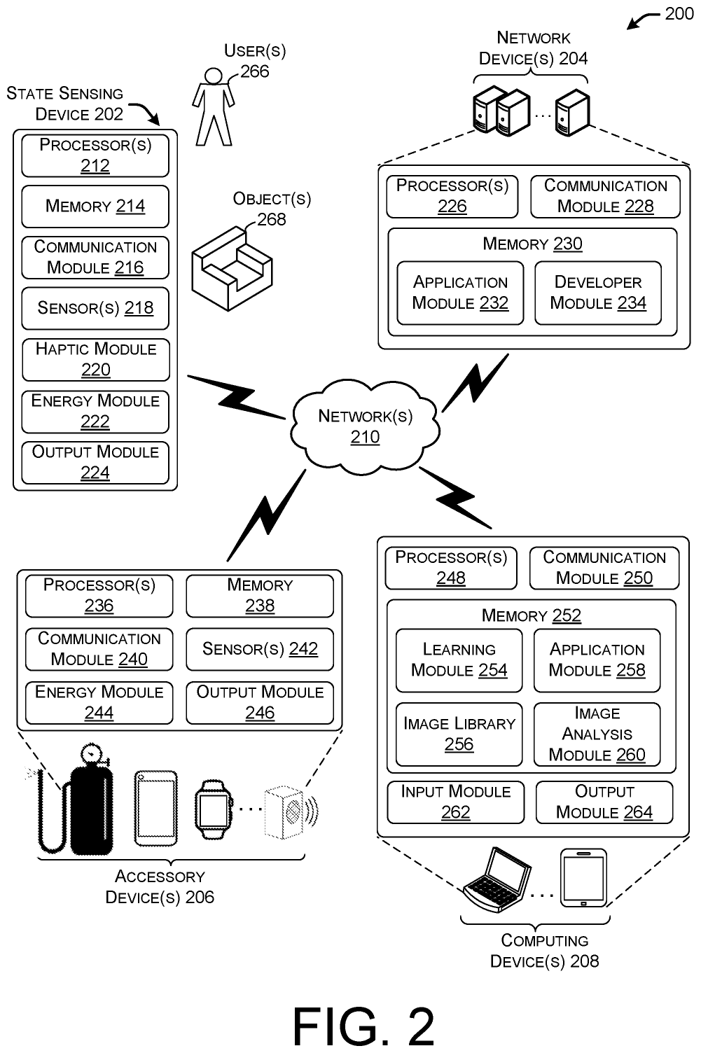

At operation 102, process 100 may include collecting and/or processing information regarding a location of the object in an environment. In an example 110, a state sensing device 112 is present in an environment 114. In this example, the environment 114 may be generally considered an interior of an airplane cabin. Example environment 114 includes an airplane seat 116 and a seat tray 118. In this case, state sensing device 112 is worn by a user 120. For illustration purposes, the location of airplane seat 116 in environment 114 may include position and/or orientation of airplane seat 116 within the three-dimensional (3D) space of the airplane cabin, a position and/or orientation of airplane seat 116 in relation to state sensing device 112 and/or user 120, and also how that 3D space of the airplane cabin relates to the world at large outside the airplane (e.g., an airport at which the airplane is parked). The location of the object may also be viewed as including a geometry of the object for purposes of this description. For instance, example 110 may include collecting information related to a geometry (e.g., size, shape) of airplane seat 116 in order to place and/or orient the airplane seat 116 inside the airline cabin, and/or in order to recognize a surface of the airplane seat 116 as an object of interest. (Note: additional detailed description regarding the one or more sensors of state sensing device 112, the information collected from the sensor(s), processing of the collected information, and/or conclusions drawn from the collected information will be provided below, relative to FIG. 2, for instance.)

Continuing with the scenario depicted in example 110, user 120 may be a member of a cleaning crew that is servicing the airplane between flights. As such, user 120 may have a disinfectant sprayer device 122 (e.g., electrostatic sprayer, aerosol sprayer). User 120 may be moving through the airline cabin, spraying airplane seat 116, seat tray 118, other surfaces, and/or performing other tasks. In this example, operation 102 may include collecting information via one or more sensors of state sensing device 112 to help determine a location of airplane seat 116. For instance, operation 102 may include determining a particular row in which airplane seat 116 is situated inside the airplane and/or a distance of a surface of airplane seat 116 from state sensing device 112. For purposes of illustration, consider that airplane seat 116 may be identified as “Seat 12B,” and that the airplane is being prepared to depart as “Flight 123.”

At operation 104, process 100 may include collecting and/or processing information regarding a condition of the object. In example 124, user 120 is spraying airplane seat 116 utilizing disinfectant sprayer device 122. Operation 104 may include sensing a visual appearance of airplane seat 116 via the one or more sensors of state sensing device 112. The visual appearance may indicate that the surface of airplane seat 116 is wetted, for instance. In example 124, a wetted surface (indicated in FIG. 1 with a shading pattern 126) of airplane seat 116 may suggest that disinfectant spray from disinfectant sprayer device 122 was discharged onto the surface of airplane seat 116.

In some examples, operation 104 may include collecting additional information and/or other types of information to help confirm a condition of the object. For instance, operation 104 may include state sensing device 112 communicating with disinfectant sprayer device 122. As such, state sensing device 112 may receive data from disinfectant sprayer device 122 indicating that disinfectant spray was discharged. Further, operation 104 may include state sensing device 112 collecting information that indicates disinfectant sprayer device 122 was pointed at airplane seat 116 (e.g., the target object) at approximately the same time that disinfectant spray was discharged from disinfectant sprayer device 122. In some examples, operation 104 may include state sensing device 112 collecting information that indicates a particular amount of disinfectant spray that was discharged from disinfectant sprayer device 122. In still further examples, operation 104 may include state sensing device 112 collecting information that indicates how long the disinfectant spray was on the airplane seat 116. For instance, state sensing device 112 may collect information that indicates a dwell time of the disinfectant spray on the airplane seat 116 before being wiped off by user 120 or another worker. In some examples, determining a dwell time may include determining thresholds, such as thresholds related to a coverage amount of an object and/or an amount of time of coverage of the object. For instance, a first threshold may relate to an amount of coverage of an object with disinfectant spray to ensure adequate disinfection of a surface. A second threshold may relate to a period of time associated with disinfectant spray in contact with the surface (e.g., contact time), again to ensure adequate disinfection. A third potential threshold may relate to a period of time that an adequate amount of coverage of disinfectant spray was in contact with the surface, ensuring that both the amount of the disinfectant spray and the dwell time are sufficient. A result of operation 104 may include that the condition of the object is “disinfected.”

At operation 106, process 100 may include fusing the location of the object (from operation 102) with the condition of the object (from operation 104). The fusion of the location of the object with the condition of the object may produce and/or help determine an overall state of the object. In example 128, data 130 located on state sensing device 112 may include any of a variety of information regarding the location and/or the condition of airplane seat 116, and/or any other information that may contribute to a determination of a state of airplane seat 116. Accordingly, operation 106 may include fusing location data and condition data relevant to airplane seat 116. In this example, a result of the fusion of information from operations 102 and 104 may be that airplane seat 116 has been disinfected. For instance, the information from operations 102 and 104 may be fused to show that a surface identified as associated with airplane seat 116 is wetted, and therefore a conclusion may be drawn (or a determination may be made) that Seat 12C of Flight 123 has been adequately disinfected.

Also illustrated in example 128 are cloud computing resources 132, and an indication (lightning bolt) that state sensing device 112 may communicate with cloud computing resources 132. In some examples, state sensing device 112 may collect information from the one or more sensors regarding the location of an object (operation 102) and/or the condition of an object (operation 104), then may send collected data 130 to cloud computing resources 132 for processing. For example, state sensing device 112 may collect information that can be used to determine a location or condition of airplane seat 116, but make not actually process the information to make the determine on board the state sensing device 112. The determination of the location and/or condition of the object may actually be made by cloud computing resources 132. In some examples, the determined location, condition, and/or state of the object may then be sent back to the state sensing device 112, may be directed to another device (e.g., for presentation to a supervisor), may be stored by cloud computing resources 132 for future reference, etc.

In some implementations, the processing of data 130 may be performed in part by state sensing device 112 (e.g., pre-processing), then sent to cloud computing resources 132 for further processing. Furthermore, some information used to determine a state of an object may be accessed and/or collected by cloud computing resources 132. For instance, cloud computing resources 132 may process data 130 together with locally-accessed information to determine the state of the object. The examples provided herein regarding the order and/or location of processing of data to determine a state of an object and/or to produce a certification of a state of an object are not meant to be limiting. Many versions of where and/or by which device such data are processed are contemplated. As noted above, additional detailed description regarding the information collected from the sensor(s), processing of the collected information, and/or conclusions drawn from the collected information will be provided below.

At operation 108, process 100 may include producing a certification of a state of the object. In some examples, a “certification” may simply include outputting a result of operation 106, such as outputting an indication of the state of the object. In example 134, an indication of a certification 136 is depicted as part of a graphical user interface (GUI) on a display 138 of state sensing device 112. In this example the GUI includes text declaring “Disinfected Seat 12B,” indicating that Seat 12B may be considered disinfected within established disinfection parameters. In other examples, related information may be provided, such as a confidence level in the disinfection, a degree of disinfection, a dwell time of the disinfectant spray on the object, etc.

Example 134 also includes representations of sound 140 and haptic feedback 142 produced by state sensing device 112. Sound or haptic feedback may be additional techniques for outputting an indication of the state of the object. For instance, operation 108 may include the state sensing device 112 emitting a sound 140 that indicates certification that the airplane seat 116 is disinfected, alerting the user 120 that he/she may proceed to spray a next surface. Similarly, operation 108 may include the state sensing device 112 producing haptic feedback 142 (e.g., vibrating) to indicate certification that the airplane seat 116 is disinfected. Indications of certification by sound or vibration may allow the user 120 to proceed with cleaning tasks with less disruption than handing a device to view a screen, for instance. In yet another example, an indication of the state of the object may be displayed or produced on an accessory device 144, such as a smart watch.

In some examples, certification may include sending indication of the state of the object to another device, such as to present an indication of the certification to a supervisor of the cleaning crew. Certification may include combining information regarding the state of the object with a state of one or more other objects. For instance, a state of an entire plane may be determined—i.e., the plane has been disinfected. Certification may include real-time and/or delayed transfer of information to other users or devices, such as notifying fellow team members that a particular task is completed, thereby informing the team of overall job progress. Stated another way, certification may include notification of aggregate job completion to team members and/or managers. In some examples, operation 108 may be performed by another device, such as cloud computing resources 132. For instance, a certification may be produced by cloud computing resources 132. An indication of the certification may then be sent to state sensing device 112 for presentation to user 120, such as via display 138, sound 140, haptic feedback 142, and/or accessory device 144.

FIG. 2 illustrates an example environment 200 including a state sensing device 202, one or more network devices 204, one or more accessory devices 206, and various computing devices 208. As shown in FIG. 2, state sensing device 202, network device(s) 204, accessory device(s) 206, and/or computing device(s) 208 may be connected by a network 210. In some implementations, some elements of FIG. 2 may be similar to elements introduced above relative to FIG. 1. For instance, state sensing device 202 may be similar to state sensing device 112. In some examples, state sensing device 202, network device(s) 204, accessory device(s) 206, and/or computing device(s) 208 may perform one or more of the operations 102-108 associated with process 100 shown in FIG. 1. Further, in some instances, features and/or operations described herein in connection with one of the devices of environment 200 may be performed by other devices of environment 200, and/or may be distributed between the various devices of environment 200. For example, processing or other operations performed by a network device 204 and/or by a computing device 208 may be performed by a state sensing device 202 and/or by an accessory device 206, and vice versa.

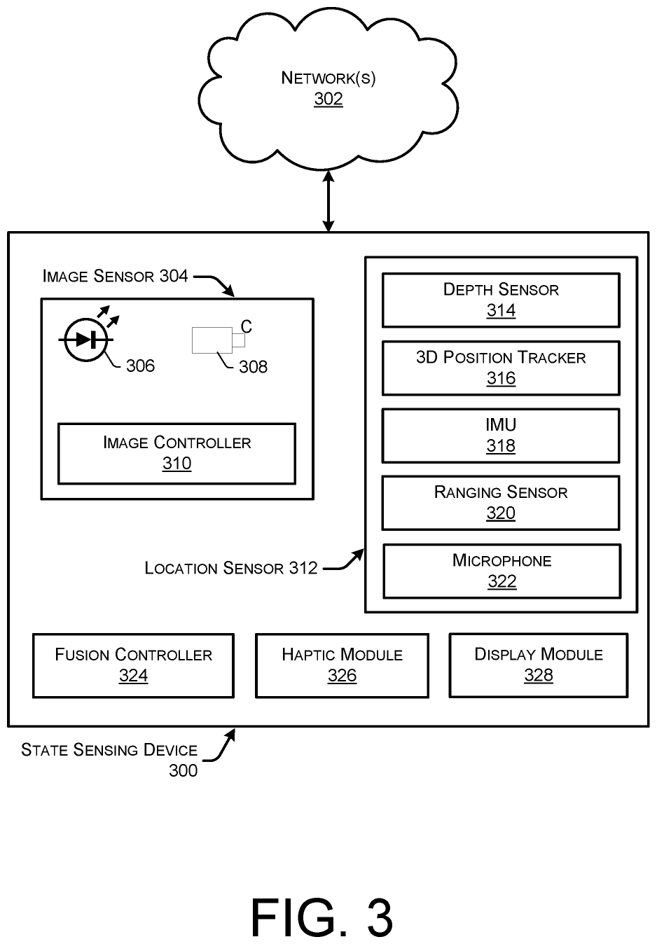

As shown in FIG. 2, state sensing device 202 may include one or more processors 212, memory 214, communication module 216, one or more sensors 218, a haptic module 220, an energy module 222 (e.g., battery), an output module 224, and/or additional modules or components. Further detail regarding a state sensing device will be provided relative to FIG. 3, below. In general, sensor(s) 218 may include any of a variety of sensors for collecting information about an environment, an object, a surface of an object, a geometry and/or condition of an object, etc.

Environment 200 also includes one or more user(s) 266, which may be associated with state sensing device 202 and/or other devices of environment 200. The one or more user(s) 266 (also referred to as a user 266) may interact with state sensing device 202, network device(s) 204, accessory device(s) 206, and/or computing device(s) 208 to perform a variety of operations discussed herein. Furthermore, environment 200 also includes one or more object(s) 268. Indeed, a purpose of the present disclosure is for state sensing device 202 to perform a variety of operations discussed herein in order to sense, collect, and/or process information that may help determine a state of object 268.

In some examples, network devices 204 may include one or more processors 226, one or more communication modules 228, and/or memory 230. Further, memory 230 may include one or more modules, such as an application module 232 and/or a developer module 234. Network device(s) 204 may be viewed as cloud computing resources, for instance.

In environment 200, accessory devices 206 may be manifest as a variety of devices that may assist in determining a state of an object and/or devices that assist in providing an indication of a determined state of an object to a user. For example, accessory devices 206 may include an electrostatic sprayer, a cell phone, a smart watch, or a speaker (shown but not designated with specificity). The example accessory devices 206 depicted in environment 200 are not meant to be limiting; a wide variety of potential accessory devices are contemplated. In some examples, accessory devices 206 may include one or more processors 236, memory 238, communication module 240, sensor(s) 242, energy module 244, and/or output module 246.

In some examples, computing devices 208 may include one or more processors 248, one or more communication modules 250, and/or memory 252. Memory 252 may include various elements such as a learning module 254, image library 256, application module 258, and/or image analysis module 260. Further, computing devices 208 may include input module 262 and/or output module 264. In some implementations, computing device(s) 208 may include, but are not limited to, any one of a variety of computing devices, such as a smart phone, a mobile phone, a personal digital assistant (PDA), an electronic book device, a laptop computer, a desktop computer, a tablet computer, a portable computer, a server computer, a wearable device, or any other electronic device.

As suggested above, in environment 200, state sensing device 202, network device(s) 204, accessory device(s) 206, computing device(s) 208 and/or other devices may communicate via one or more network(s) 210. In some instances, network 210 may represent one or more wired or wireless networks, such as the Internet, a Mobile Telephone Network (MTN), or other various communication technologies. In some instances, network 210 can include any WAN or LAN communicating via one or more wireless protocols including but not limited to RFID, near-field communications, optical (IR) communication, Bluetooth, Bluetooth low energy, ZigBee, Z-Wave, Thread, LTE, LTE-Advanced, New Radio (NR), WiFi, WiFi-Direct, LoRa, Homeplug, MoCA, Ethernet, etc. In some instances, network 210 may include one or more mesh networks including state sensing device 202, network device(s) 204, accessory device(s) 206, computing device(s) 208, and/or other devices.

In some examples, the processor(s) introduced above (e.g., processor(s) 212, 226, 236, 248) may be a single processing unit or a number of units, each of which could include multiple different processing units. The processor(s) can include one or more microprocessors, microcomputers, microcontrollers, digital signal processors, central processing units (CPUs), graphics processing units (GPUs), security processors (e.g., secure cryptoprocessors), and/or other processors. Alternatively, or in addition, some or all of the techniques described herein can be performed, at least in part, by one or more hardware logic components. For example, and without limitation, illustrative types of hardware logic components that can be used include Field-Programmable Gate Arrays (FPGAs), Application-Specific Integrated Circuits (ASICs), Application-Specific Standard Products (ASSPs), state machines, Complex Programmable Logic Devices (CPLDs), other logic circuitry, systems on chips (SoCs), and/or any other devices that perform operations based on software and/or hardware coded instructions. Among other capabilities, the processor(s) can be configured to fetch and/or execute computer-readable instructions stored in the memory (e.g., memory 214, 230, 238, 252).

In some examples, the memory introduced above (e.g., memory 214, 230, 238, 252) may include one or a combination of computer-readable media. As used herein, “computer-readable media” includes computer storage media and communication media. Computer storage media (e.g., also referred to as non-transitory computer-readable media) may include volatile and non-volatile, removable and non-removable media implemented in any method or technology for storage of information, such as computer-readable instructions, data structures, program modules, or other data. Computer storage media may include, but is not limited to, Phase Change Memory (PCM), Static Random-Access Memory (SRAM), Dynamic Random-Access Memory (DRAM), other types of Random-Access Memory (RAM), Read-Only Memory (ROM), Electrically Erasable Programmable ROM (EEPROM), flash memory or other memory technology, Compact Disc ROM (CD-ROM), Digital Versatile Discs (DVD) or other optical storage, magnetic cassettes, magnetic tape, magnetic disk storage or other magnetic storage devices, or any other medium that can be used to store information for access by a computing device.

In contrast, communication media may include computer-readable instructions, data structures, program modules, or other data in a modulated data signal, such as a carrier wave. As defined herein, computer storage media does not include communication media.

In some examples, a communication module (e.g., communication module 216, 228, 240, 250) may include functionality to receive wired or wireless data from network(s) 210 and/or from one or more of state sensing device 202, network device(s) 204, accessory device(s) 206, computing device(s) 208 and/or additional computing devices. In some instances, the communication module can receive data in accordance with one or more transmission protocols, such as HTTP, HTTPS, Bluetooth, Bluetooth low energy, Wi-Fi, etc. In some instances, the communication module may monitor a strength of a wireless signal associated with state sensing device 202 and/or an accessory device 206 in conjunction with other data to determine a location of state sensing device 202 (e.g., using a received signal strength indicator (RSSI) or a received signal power).

Input module 262 of computing device(s) 208 may include various input devices such as an imaging device, one or more microphones, a touch display, one or more proximity sensors, etc. In some instances, the input module 262 may further include sensors such as one or more accelerometers, gyroscopes, barometers, temperature sensors, GPS sensors, light sensors, etc. As such, various components of state sensing device 202 may be viewed as part of an input module of state sensing device 202, such as sensor(s) 218. In some examples, output module(s) (e.g., output module 224, 246, 264) may include one or more output devices generating audible output (e.g., via a speaker), visual output (e.g., via a display), and/or haptic feedback (e.g., vibration motors). The presence or absence of a specific input module, output module, and/or other input/output component as depicted in FIG. 2 is not meant to be limiting.

In some examples, the energy modules introduced above (e.g., energy module 222, 244) may include one or a combination of a battery, capacitor, supercapacitor, ultracapacitor, fuel cell, electrochemical power supply, spring, flywheel, solar cell, solar panel, etc. Further, an energy module may include one or more connectors configured to receive power from an external power source, such as via an external battery or via power provided from a utility. Such a connector may be used to recharge a battery of the energy module, for instance.

As used herein, the term “module” is intended to represent example divisions of software and/or firmware for purposes of discussion, and is not intended to represent any type of requirement or required method, manner or organization. Accordingly, while various “modules” are discussed, their functionality and/or similar functionality could be arranged differently (e.g., combined into a fewer number of modules, broken into a larger number of modules, etc.). Further, while certain functions are described herein as being implemented as software modules configured for execution by a processor, in other embodiments, any or all of the functions can be implemented (e.g., performed) in whole or in part by hardware logic components, such as FPGAs, ASICs, ASSPs, state machines, CPLDs, other logic circuitry, SoCs, and so on.

In general, any of network device(s) 204, computing device(s) 208, and/or accessory device(s) 202 may include functionality to send and/or receive data associated with state sensing device 202 to determine a state of an object 268 and/or to notify a user 266 of the state of the object 268.

FIG. 3 shows an illustrative functional block diagram of an additional example state sensing device 300. In some implementations, components of state sensing device 300 may be similar to components introduced above relative to state sensing device 112 (FIG. 1) and/or state sensing device 202 (FIG. 2). As shown in FIG. 3, state sensing device 300 may communicate with other devices via network(s) 302, which may be similar to network(s) 210 (FIG. 2).

State sensing device 300 may include an image sensor 304. Image sensor 304 may include various components, such as a light emitter 306, a camera 308, and/or an image controller 310. State sensing device 300 may also include a location sensor 312. Location sensor 312 may include various components, such as a depth sensor 314, a three-dimensional (3D) position tracker 316, an inertial measurement unit (IMU) 318, a ranging sensor 320, and/or a microphone 322. Any of the components listed above may be viewed as input components and/or part of an input module, for instance. State sensing device 300 may also include a fusion controller 324. In some implementations, image controller 310 and/or fusion controller 324 may include and/or share one or more processor(s) and/or memory (such as processor(s) 212 and/or memory 214, described above relative to FIG. 2). State sensing device 300 may also include a haptic module 326, which may be similar to haptic module 220 (FIG. 2). State sensing device 300 may also include a display module 328. In other examples, a haptic module and/or display module may be viewed as part of an output module (e.g., output module 224 of FIG. 2).

In general, image sensor 304 of state sensing device 300 may be used to collect information regarding a condition of an object, similar to operation 104 of process 100 of FIG. 1. Further, location sensor 312 of state sensing device 300 may be used to collect information regarding a location of an object, similar to operation 102 of FIG. 1. Fusion controller 324 may perform tasks related to fusing the location of the object (from operation 102) with the condition of the object (from operation 104), similar to operation 106 of FIG. 1. Finally, haptic module 326 and/or display module 328 may participate in providing feedback to a user of state sensing device 300, which may be similar to some aspects of operation 108 of FIG. 1. Note that information collected by image sensor 304 and/or location sensor 312 may also be sent to one or more other computing device(s) via network(s) 302, such as for data processing, reporting, information storage, etc.

In some examples, image sensor 304 of state sensing device 300 may be viewed as assisting classification of the object. Stated another way, image sensor 304 may collect information about the object that helps inform a classification that is associated with the state of the object. The classification may be performed relative to two-dimensional (2D) space, or regarding a 2D image. For instance, the camera 308 may capture an image that includes the object, and a classification of at least part of the image that includes the object may be performed using the image. In some examples, the image sensor 304 can be used to capture sensor data to determine and/or confirm a location of the device 300 in the environment. For example, the image sensor 304 can capture a seat number on an airplane or a room number in a hotel, which can be used in addition to other techniques discussed herein to determine an initial position or to increase a confidence of a location in an environment.

Camera 308 of image sensor 304 may represent one or more of a variety of cameras for collecting image data from an environment, or may include a combination of types of cameras. In some implementations, camera 308 may be a visible light camera (e.g., red green blue (RGB)), an ultraviolet (UV) camera, an infrared camera, etc. Light emitter 306 may be capable of emitting light into an environment, such as exciting light. The emitted light may be visible spectrum, infrared (IR) spectrum, UV spectrum, etc. For instance, light emitter 306 may be manifest as one or more UV light emitting diodes (LEDs). In some scenarios, an appearance of an object may change when it is illuminated with a different type of light. Therefore, light emitter 306 may be used in a coordinated fashion with camera 308 to capture different images of an object to provide information regarding a condition of the object. For instance, in one example scenario where light emitter 306 consists of two UV LEDs, the LEDs may be turned off, and camera 308 may capture a first image of the object. Then the LEDs may be turned on, and camera 308 may capture a second image of the same object. A difference in appearance of the object between the first image and the second image may help make a determination about the condition of the object.

Consider the scenario introduced relative to environment 114 in FIG. 1. In this scenario, an airplane seat is being disinfected. Also, a determination is to be made regarding whether or not the airplane seat is sufficiently disinfected. In this scenario, the disinfectant sprayer device 122 may be a commercial-grade electrostatic sprayer. Electrostatic sprayers may apply disinfectant relatively heavily. The disinfectant liquid may soak a surface to which it is applied, like a light rain, rather than a relatively thin aerosol spray. In this scenario, the image sensor 304 may be able to collect information that helps answer whether a surface of the airplane seat is covered with a mist or small beads of water, whether the surface is wetted, etc. In some cases, exciting light emitted (e.g., from light emitter 306) from a known angle relative to a surface of an object may be expected to be reflected back in a certain way (e.g., to camera 308). For instance, a wetted airplane seat may reflect light differently than a non-wetted airplane seat.

In another scenario, the disinfectant liquid may have known optical properties that are detectable with the image sensor 304. Some disinfectant and/or other types of cleaning fluids may have a greater of lesser degree of a particular optical property. For instance, some fluids may be visible under UV light, but less detectable (or not detectable) under visible light. Therefore, an object may be sprayed with a disinfectant liquid that is detectable under UV light, and the camera 308 may capture images of the object with UV light on and off via light emitter 306. The result may be a difference in appearance of the object under UV light, which may be an indicator of disinfection.

Various additional or alternative properties, features, and/or characteristics are contemplated for detection via image sensor 304 in order to help determine a state of an object. For instance, image sensor 304 may be able to sense dirt and/or dust that requires cleaning. Image sensor 304 may be able to sense damage that requires attention. For example, image sensor 304 may be able to help determine that a seat tray on an airplane has been broken and may need repair. A broken object may be detected through polygon analysis of images, determining that a shape of an object has changed, for instance. In still another example, a texture or pattern in an image may appear differently under UV light. The examples of differentiation for the state of an object suggested herein are not meant to be limiting; many types of field discriminators are contemplated.

Image controller 310 of image sensor 304 may be capable of directing the camera 308 to capture an image and/or directing the light emitter 306 to illuminate an environment, in some cases. Furthermore, image controller 310 may be capable of performing part or all of processing of the data associated with an image captured by camera 308. Stated another way, computer instructions stored on the image controller 310 may provide edge computing and/or local computing before sending a result on to a next system, such as fusion controller 324 or another device. Such local computing may potentially help minimize bandwidth requirements related to transferring data, such as image data, to another device. Local computing may also potentially help inform local decisions that drive local actions, saving time. For instance, subsequent images may need to be captured in a scenario where a determination is not able to be completed, or not completed with sufficient confidence, etc. In this instance, local computing may be able to quickly determine that another image should be captured, perhaps with or without light emitter 306 turned on, and direct the image sensor 304 to capture another image via camera 308, more quickly resolving the issue and/or producing a more confident determination of the state of the object.

In some examples, image controller 310 of image sensor 304 may use a comparison to help make a determination associated with a state of a surface of an object. For example, image controller 310 may compare a presently captured image to an expected image of the surface of the object. In some examples, image controller 310 may use a model and/or algorithm to help make a determination associated with a state of a surface of an object. For instance, a machine learned model may be used to help determine a state of a surface of an object.

In some implementations, an image captured by camera 308 may be passed through one or more classifiers. For example, a sequence of classifiers may be used as a pipeline (e.g., linear pipeline) to process an image. A classifier may apply a test, such as a comparison with an expected characteristic of the image. A classifier may use one or more parameter(s) and/or threshold(s) in the test, to help determine how the image compares to an expected characteristic. In some examples, a classifier may have a relatively large number (e.g., 100) of parameters and/or thresholds. The parameter(s) and/or threshold(s) may be determined ahead of time through machine learning.

To support machine learning for image classification for the purpose of determining a state of an object, training data may be acquired that relate to expected objects or scenes. For instance, relating to the example environment 114 of FIG. 1, training data may include images of sprayed airplane seats and unsprayed airplane seats, clean seat trays and dirty seat trays, intact seat trays and broken seat trays, etc. Training data images may be captured using a state sensing device (e.g., state sensing device 112, 202, 300). Training data images may also be captured using any of a variety of other devices, and/or image databases may be accessed for training data. In another example, ad hoc video and/or other images may be supplied by an airline or hotel company (e.g., a video or image detailing standard operating procedures or for training employees), or cell phone pictures may be collected from a variety of users. Training data may be kept in an image database and/or library. Training data may be annotated, such as in a supervised data set. In some examples, annotation may apply to overall surfaces and/or whole images. In other examples, annotation may include per pixel and/or sub-image annotation.

In some examples, the training data may be used to help determine more successful classifiers, parameters, and/or thresholds. Stated another way, training data may help determine which classifiers, parameters, and/or thresholds are more likely to produce a correct and/or relatively highly confident determination of a state of an object. In some examples, a large number (e.g., a million) of training data images may be processed through a large number (e.g., another million) of combinations of different potential parameters and/or thresholds for a classifier to help determine which training data image and/or combination produces a (potentially) best score. For instance, which training data image and/or combination produces the best classification of a surface being wetted. Many types of processing and/or machine learned models are contemplated for processing training data, such as a support vector machine (SVM), cluster analysis, a neural network (e.g., a convolutional neural network, a recurrent neural network, a graph neural network), etc.

Referring again to FIG. 2, in some examples, the algorithm and/or machine learned model may be produced (e.g., trained) by one or more of computing devices 208 and/or network devices 204. For instance, learning module 254 of a computing device 208 may perform machine learning functions, using training data stored in image library 256. Further, image analysis module 260 may be used for image annotation, other tasks involved with helping to determine parameters and/or thresholds for classifiers, etc. Alternatively, developer module 234 of a network device 204 may be used to perform machine learning functions. Once an algorithm and/or machine learned model is produced using the training data, the algorithm and/or machine learned model may be stored on the state sensing device 300 (referring again to FIG. 3). For example, the algorithm and/or machine learned model may be part of firmware within image controller 310. A newly captured image may then be passed through the pipeline of classifiers, and image controller 310 may use the firmware to make a determination of a state of the surface of the object, such as whether or not an airplane seat has been sprayed with disinfectant.

As suggested above, simply declaring that a surface of an airplane seat has been disinfected may not be helpful without knowing where in the world the airplane seat is located, and/or at what point in time the airplane seat was disinfected. Therefore, state sensing device 300 may also include location sensor 312. As introduced above, location sensor 312 may include various components, such as a depth sensor 314, a three-dimensional (3D) position tracker 316, an inertial measurement unit (IMU) 318, a ranging sensor 320, and/or a microphone 322.

In some examples, depth sensor 314 may be a 3D scanner. A 3D scanner may provide a 3D point cloud. Stated another way, a 3D scanner may be capable of producing a 3D mesh constructed of distances from the 3D scanner to various points of objects in an environment. In some examples, a region of interest in the image may be clipped out for processing. For instance, a region that is expected to contain an object of interest may be clipped out.

In some examples, 3D position tracker 316 may be able to provide information suggesting a location. For example, 3D position tracker 316 may be able to sense where in a room state sensing device 300 is located. More specifically, 3D position tracker 316 may be able to determine that state sensing device 300 is located near Row 12 in an airplane cabin, in a bathroom of a hotel room, etc. Information from 3D position tracker 316 may be combined with GPS data or other image data to determine where in the world state sensing device 300 is located. For instance, that state sensing device 300 is located near Row 12 in the airplane cabin of Flight 123, in the bathroom of Room 201 of a specific hotel, etc.

In some implementations, 3D position tracker 316 may be manifest as one or more cameras. For instance, multiple cameras may be able to produce stereoscopic and/or binocular images that may be processed to provide depth information. In one example, 3D position tracker 316 may comprise two grayscale, fish-eye cameras. Further, 3D position tracker 316 (and/or another component) may perform Harris corner detection, which may include processing the images for characteristic points (e.g., corners, Harris corners). The image(s) may be searched to find characteristic points, then common characteristic points may be located in paired image sets from the coordinated cameras. Binocular fusion of the two characteristic point sets may help determine an amount of motion between image frames. A frame frequency captured by 3D position tracker 316 may be 200 times per second, for instance.

Information from the 3D position tracker 316 may then be oriented using information from IMU 318. IMU 318 may be able to produce information regarding orientation (e.g., yaw) of state sensing device 300, such as whether the device is pointed up, down, east, south, etc. Therefore, IMU 318 and 3D position tracker 316 together may provide a spatial offset from a starting point of observation by state sensing device 300, as well as an orientation of state sensing device 300. Further, a 3D position of state sensing device 300 over time may be found through the coordinated efforts of 3D position tracker and IMU 318. The culmination of this position and/or movement tracking may be an understanding of where in space the 3D mesh from depth sensor 314 is located.

In some examples, ranging sensor 320 may be able to provide a relatively precise measurement of a distance of an object from state sensing device 300. For example, ranging sensor 320 may be able to measure a distance of state sensing device 300 to a center point within a range of view (e.g., field of view) of one or more cameras of state sensing device 300. In general, ranging sensor 320 may be able to provide a more accurate distance measurement than depth sensor 314 (at least to a single point of the range of view). A distance measured by ranging sensor 320 may be precise to approximately one millimeter, for instance. In some implementations, ranging sensor 320 may be manifest as a single point structured light range detector. Other technology examples that may be associated with ranging sensor 320 may include time of flight (ToF), RGB-depth, etc. A single point ranging sensor 320 may be used to reduce cost, while in some examples a ranging sensor 320 may measure distances to multiple points.

Additionally, location sensor 312 may include microphone 322. Microphone 322 may be included to record sound, such as sound that provides information about an environment, sound that includes a voice command, etc. In some implementations, a speaker may also be present (not shown). In some examples, the microphone 322 (and/or speaker) may be used to send and/or receive ultrasonic sounds to further identify a location and/or velocity of the state sensing device 300, such as by using frequency and/or phase measurement techniques (e.g., determining a Doppler shift of the sound).

Data from the various components of location sensor 312 may be processed to provide a 3D view of an environment in which an object is located. In some cases, the data for the components may be processed by the location sensor 312. For example, location sensor may have access to processing and/or memory capability, such as processor(s) 212 and/or memory 214 shown in the example state sensing device 202 in FIG. 2. Note that information collected by location sensor 312 may be combined with additional information, such as map data, in order to determine a location of an object and/or a 3D view of an environment.

Processing of the 3D data collected by the components of location sensor 312 may include segmentation in some cases. For example, a surface of an object of interest (e.g., target) may be segmented out from a 3D view of an environment. For instance, state sensing device 300 may be located two meters away from a desk. A portion of a 3D map associated with the desk may be segmented out from the surrounding room. More specifically, the desk may be considered a point of interest, and everything that is not the point of interest may be filtered out. In this manner, the desk may be analyzed independently. In some examples, statistical pattern recognition of the segmented portion of the field of view (FOV) may be used.

One example method associated with segmentation that may be performed by location sensor 312 is connected component analysis. For instance, a depth map may provide a depth to each pixel in the 3D view of the environment. The depth map may be analyzed to find connected pixels, resulting in a pixel map. Connected component analysis may then be performed on the pixel map. In another example, the depth map may be transformed into a tensor map. In a tensor map, a vector points along a direction of greatest change of distance from the camera (e.g., depth sensor 314). A vector associated with each pixel may be coordinated to neighboring pixels to show how each pixel is related. Connected component analysis may then be applied to the resultant tensor field (rather than the pixel map, as above). A connected component algorithm may or may not be associated with training. Connected component analysis may include setting one or more thresholds. In some instances, one or more thresholds may be set through trial and error.

In some cases, applying connected component analysis to a tensor field may generate a more accurate segmentation result. For instance, using a tensor field where a surface that is oriented relatively obliquely with respect to the camera (e.g., depth sensor 314) may generate a more accurate segmentation result than using a pixel map. Stated another way, using the tensor field may be an improvement over using the pixel map in some cases, such as where a surface of interest is not particularly perpendicular to the camera view. Therefore, connected component analysis using a tensor field may represent an improved method for segmentation related to determining a state of an object. Such an improvement may significantly impact scenarios in which use of a state sensing device 300 is envisioned. For example, a user of a state sensing device 300 moving about an airline cabin, a hotel room, a conference room, may not always be conveniently perpendicular to surfaces of interest. Consider an airplane cabin, where space is typically constrained. A state sensing device 300 is likely to have a relatively oblique view of surfaces such as airplane seats, seat trays, etc. Similarly, at least part of a curved sink (e.g., a hotel bathroom sink), may always be oblique relative to a state sensing device 300. Therefore, methods for determining a state of an object that are robust to viewing surfaces at oblique angles may be more successful in general.

State sensing device 300 may also include fusion controller 324. As noted above, fusion controller 324 may perform tasks related to fusing the location of an object provided by the location sensor 312 with the condition of the object provided by the image sensor 304. For example, image sensor 304 may collect and process data related to an image of a target surface and determine whether the target surface is wet and/or shows other characteristics that would indicate the target surface has been disinfected. Fusion controller 324 may receive a result (e.g., classification) from the image sensor 304 indicating that the target surface is disinfected. Fusion controller 324 may also receive a result from location sensor 312 that indicates where the target surface is located in the world. In some examples, fusion controller 324 may fuse the results from image sensor 304 and location sensor 312 and send the fused result to another device, via network(s) 302.

In some examples, fusion controller 324 may perform relatively little calculation regarding the data from the image sensor 304 and location sensor 312. For instance, the fused result may be sent to cloud computing resources for cataloging, storage, visual review, etc., such as by application module 232 of a network device 204 (FIG. 2). More specifically, fusion controller 324 may gather the data from image sensor 304 and location sensor 312 and send the data to cloud computing resources. The cloud computing resources may review the data to make the determination that a particular airplane seat on a particular flight was disinfected, and may be certified as such. The certified state of the object may then be returned to the state sensing device 300 in some cases, such as returned to the fusion controller 324. Alternatively, the fused result may be sent to a computing device 208 for cataloging, storage, visual review, etc., such as by application module 258. In some examples, fusion controller 324 may be viewed as a central repository for data from the various components of the image sensor 304 and/or the location sensor 312. Note that fusion controller 324 may also receive/forward and/or process information from additional types of sensors (e.g., accelerometers, barometers, gyroscopes, pressure sensors, magnetometers, capacitive sensors, etc., not shown) of state sensing device 300. Fusion controller 324 may also save some or all of the associated data locally.

In some implementations, state sensing device 300 may include a display module 328, which may also generate a visual display using some or all of the associated data. In some cases, fusion controller 324 may generate the visual display, and display module 328 may simply cause the visual display to be presented. For instance, fusion controller 324 may composite a graphical user interface (GUI), which may be presented via a display of state sensing device 300 (e.g., GUI 136 and display 138 of state sensing device 112 in FIG. 1). In one example, fusion controller may combine image data from image sensor 304, location data from location sensor 312, and a certified disinfected determination from cloud computing resources via network(s) 302 to generate a visual display.

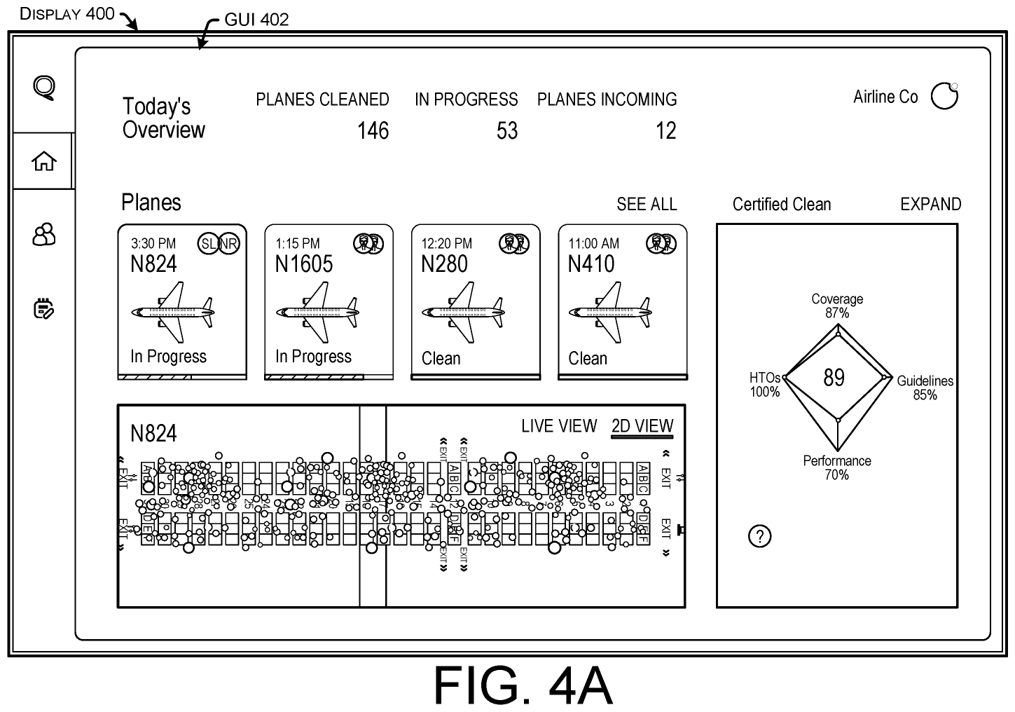

In some implementations, fusion controller 324 may send a visual display to another device for presentation, such as a computing device 208 (FIG. 2). In other implementations, application module 258 may generate a visual display for presentation to a user of a computing device 208. FIGS. 4A and 4B show illustrative examples of a display of information generated by one or more state sensing devices (e.g., state sensing device 112, 202, 300). FIGS. 4A and 4B may include an example display 400, which may be a display of a computing device 208. FIGS. 4A and 4B may include example GUIs 402 and 404, which may be generated at least in part from information that was collected using a state sensing device(s).

FIGS. 4A and 4B may represent results related to the state of one or more objects. The display may be intended for a manager or supervisor may wish to review the work of an employee and/or cleaning crew, or determine whether a particular airplane is prepared for a new group of passengers to board. In another example, the display may be intended for a customer interested in seeing that a surface was sprayed/disinfected. The customer may wish to review the results and/or a schedule of disinfection. In some examples, evidence of spraying paired with a timetable may constitute a binding certification that a company that manages the environment has appropriately disinfected the space. As such, an example as shown in FIG. 4A or 4B, or another version of the results, may be presented on a computing device 208 of a manager, or on a mobile device of a customer, etc.

A wide variety of visual display types are contemplated for outputting results generated at least in part from information that was collected using a state sensing device(s). More or less of the information that is collected may be presented. For example, a disinfection determination may be made on a per pixel basis relative to an image. As such, each pixel may be classified as sprayed or not sprayed with a disinfectant spray (e.g., by image controller 310 of FIG. 3). This per pixel classification information may be provided to network device(s) 204 (FIG. 2). Application module 232 of network device(s) 204 may analyze the per pixel classification information to determine that a threshold number of pixels of an airplane seat, or a threshold percentage of the surface area of the airplane seat, has been sprayed. Therefore, application module 232 may conclude that the airplane seat has satisfactorily disinfected, and generate a certification of disinfection for the airplane seat. However, subsequently, application module 232, fusion controller 324, and/or display module 328 may present more or less information to a user of the state sensing device, or to a user of a computing device 208. In this scenario, potential display information could include any of the image information (e.g., a view of the airplane seat), disinfection information layered onto an image of the airplane seat (e.g., a color-coded image indicated areas of the airplane seat that were disinfected), a rotatable 3D model of the airplane seat, simple text indicating that the airplane seat was disinfected or not, text indicating that the airplane has been certified as disinfected, a confidence level in the disinfection, etc. In some implementations, a customer may request and be provided more or less information. In some implementations more or less information may be provided at a future date, such as for evidence in a court case. In the example of a color-coded image indicated areas that were disinfected, pixels in which disinfection was detected may be mapped back onto a 2D or 3D model as a colored region.

As introduced above, various other types of output are contemplated, such as via haptic module 326. Haptic module 326 may include vibration motors or other componentry for physically alerting a user. For instance, state sensing device 300 may vibrate to alert a user that a disinfection attempt was unsuccessful. In this manner, haptic module 326 may be viewed as providing guidance that can direct the user. Thus, haptic module 326 may provide feedback, direction and/or fine-tuned control to an employee. In some examples, haptic, visual, or sound feedback may be tied into other things in an environment. For instance, a state sensing device in a factory may communicate with a nearby robot, providing safety feedback regarding movement of equipment in the factory, and may alert a user to potential danger. Additionally or alternatively, an output module of a state sensing device (e.g., output module 224 of state sensing device 202 of FIG. 2) may provide information to a user that is not related to the state of an object. For instance, the output module 224 may inform the user that a job has been completed, or that a new task has been prioritized for the user, etc.

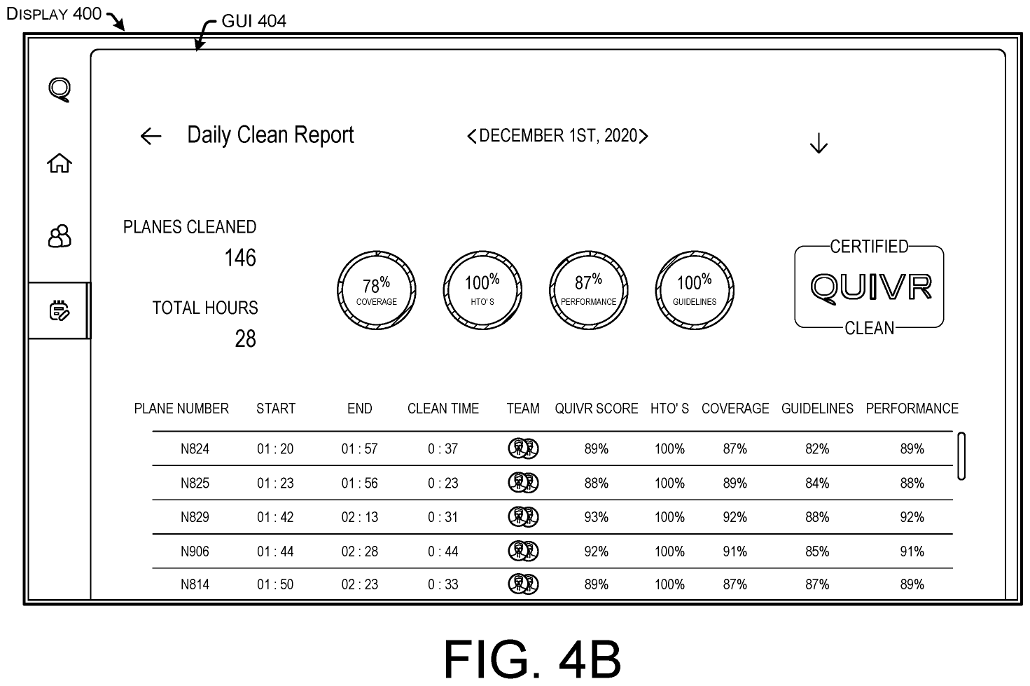

FIGS. 5-8 show illustrative examples of various additional state sensing devices. FIGS. 5-8 illustrate a variety of ways a state sensing device may be worn and/or mounted, for instance. FIG. 5 is a front elevational view that includes an example state sensing device 500 worn be a user 502. As shown in FIG. 5, a harness 504 or other equipment may be employed. FIG. 5 includes several cameras and/or other sensors on state sensing device 500 (shown but not labeled with specificity to avoid clutter on the drawing page). As such, the sensors are directed outward from user 502 while state sensing device 500 is worn in harness 504, providing an appropriate view for the sensors.

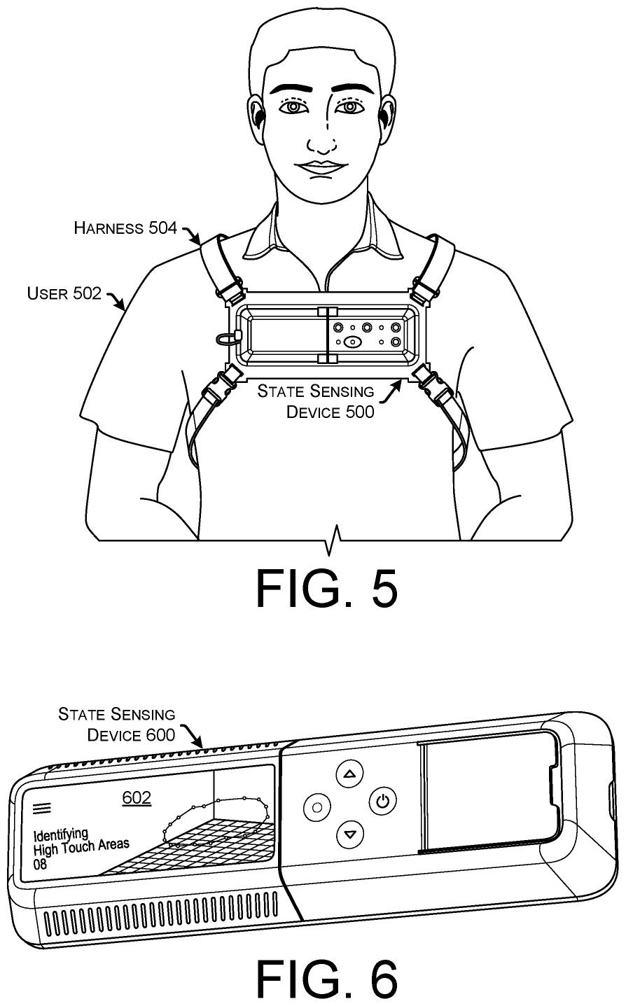

FIG. 6 is a perspective view that includes an example state sensing device 600 with a display 602. The display 602 may be any of a variety of display screens for presenting visual information, including an interactive touch display. In some examples, the display 602 may be located on an opposite side of state sensing device 600 from other components of state sensing device 600, such as sensors (e.g., cameras). For instance, the view shown in FIG. 6 may represent a “back” side of state sensing device 600 with the display 602 visible, while the view shown in FIG. 5 may represent a “front” side of state sensing device 600 with sensors visible.

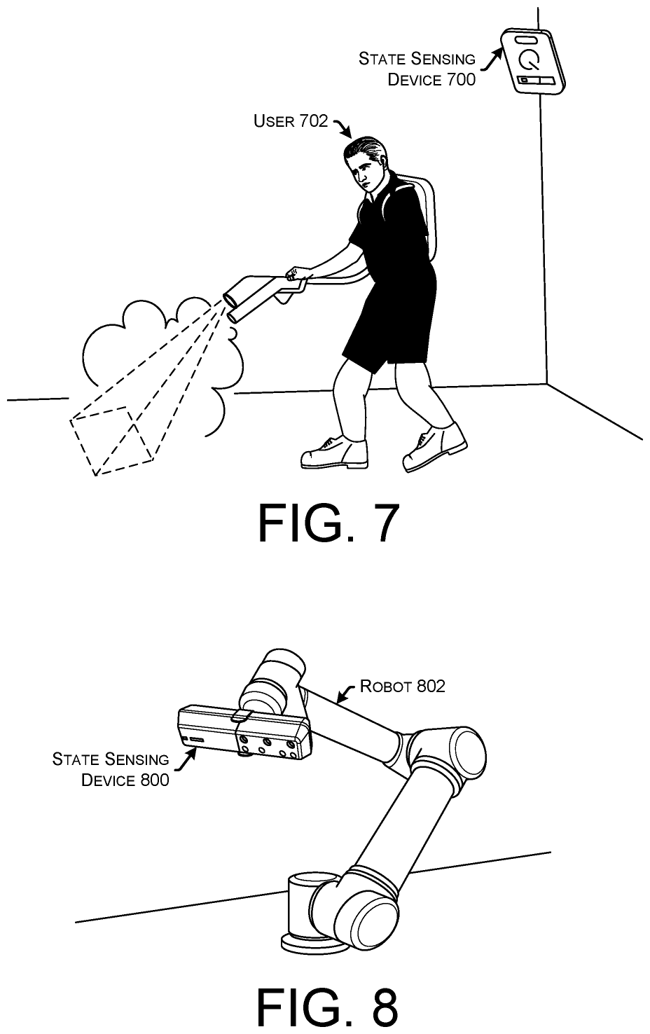

FIG. 7 is a perspective view that includes an example state sensing device 700 in an environment with a user 702. As shown in FIG. 7, state sensing device 700 may be mounted in a room, such as on a wall. In this example, user 702 is cleaning in the room. In some implementations, user 702 may be receiving feedback from state sensing device 700. For example, an output module of state sensing device 700 (e.g., output module 224 of FIG. 2) may be providing feedback regarding progress of the cleaning task based at least in part on information sensed by state sensing device 700. The feedback may be provided to user 702 via an accessory device (e.g., an accessory device 206 of FIG. 2).

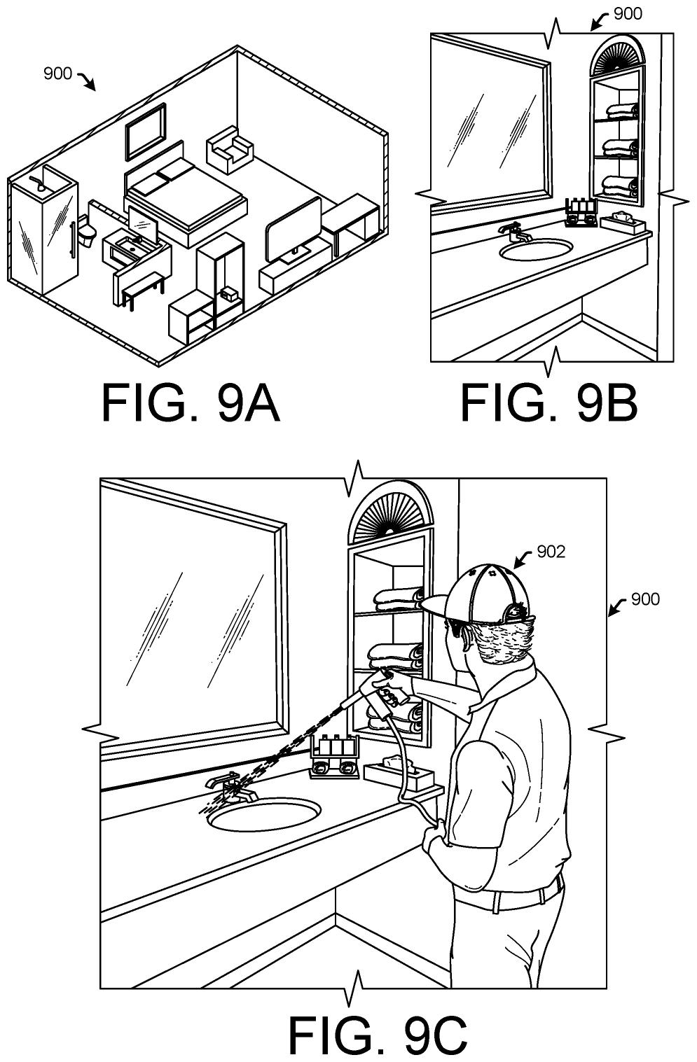

FIG. 8 is a perspective view that includes an example state sensing device 800, which may be located in a factory, workshop, repairshop, laboratory, etc. As shown in FIG. 8, state sensing device 800 may be mounted on an arm of a robot 802 or some other type equipment, which may or may not be mobile. State sensing device 800 may be used to classify products as assembled, repaired, etc. A wide variety of applications are possible in a commercial and/or industrial type setting. In one example, state sensing device 800 may assist with classification of objects that are very small and may be difficult for a person to view. Note that in this instance, a camera associated with state sensing device 800 may be different than a camera associated with state sensing device 700 (FIG. 7). For instance, a camera associated with state sensing device 800 may need a different focal length and/or a finer focus than a camera associated with state sensing device 700. Features and/or inputs (e.g., field of view, range, frame rate, thresholds, parameters, sensor durations, etc.) of either a sensor or the software used to fuse the sensor information may be designed and/or adjusted to suit an environment in which any particular state sensing device is meant to be deployed.

In other examples, a state sensing device may assist with classification of objects that are moving very quickly, and may be difficult for a person to record pertinent information in a timely fashion. For instance, parts, robots, and/or other equipment may be moving very quickly, and a person may not be able to track all of the movement of all of the equipment in real time. A state sensing device maybe be able to collect and process information faster than a human, helping ensure safety, task completion, etc. In other settings, a state sensing device may be able to detect a leak or spill. For instance, a state sensing device may be able to check for leaking water or gas from a pipe, check for leaking oil or coolant from an engine, etc.

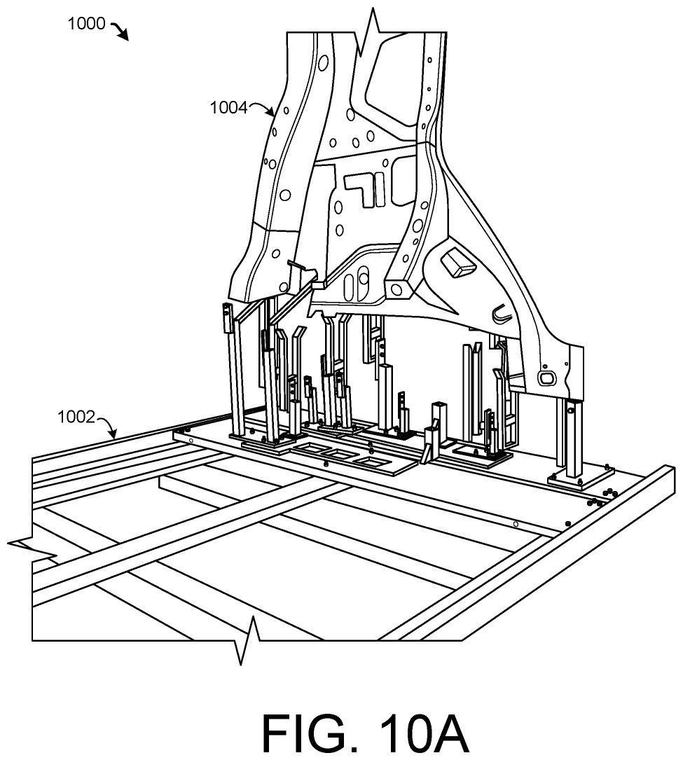

FIGS. 9A-9C include various perspective views of an example environment 900. FIG. 9A is a partial cutaway perspective view. In this example, environment 900 is a hotel room. FIGS. 9B and 9C are perspective views of a portion of environment 900. More specifically, FIGS. 9B and 9C show perspective views of a bathroom of environment 900. FIGS. 9A-9C illustrate an example scenario in which a hotel room is expected to be disinfected between guest stays. As shown in FIG. 9C, an employee 902 may be disinfecting the sink area of the bathroom of the hotel room. Although not visible in FIG. 9C, employee 902 may be wearing a state sensing device, similar to state sensing device 500 in FIG. 5.

In some implementations, techniques associated with object state determination may include predetermining areas of an environment that may be targeted by a state sensing device. Similarly, techniques associated with object state determination may include training employees on tasks that may be performed while using a state sensing device. An example of predetermining areas of an environment may include a supervisor designating areas of a hotel room that may need to be sprayed. In the example shown in FIG. 9A, the bed surface (e.g., bedspread), bathroom sink area, and/or any of a variety of other surfaces in environment 900 may be selected for disinfection. In this example, the supervisor may walk into the hotel room and direct a state sensing device towards different objects, such as the bed surface and bathroom sink area, as part of a training process. Subsequently, when the state sensing device “sees” the room, the state sensing device may be able to match objects relative to images that were captured in the training process. Note that in the subsequent instance, the same state sensing device may be used or a similar state sensing device that has access to information from the training process may be used. Further, the (same or similar) state sensing device may be able to target objects that were designated by the supervisor as objects and/or points of interest (e.g., the bedspread, the bathroom sink).

Predetermining areas of an environment may include specifying boundaries for objects, points, and/or surfaces of interest (e.g., a relevant surface of interest). For example, a supervisor may wish to designate a bathroom sink as object of interest. A bounding region of the bathroom sink may be limited to the sink bowl and faucet handles, or may include the entire sink countertop area. Boundary specification may be performed by the supervisor during the training process. For instance, the supervisor may walk into environment 900 with a state sensing device. The supervisor may be able to view, via a display, an object that the state sensing device is sensing. The supervisor may be able to approve the object and/or boundaries of the object. The supervisor may also be able to reject the object and/or boundaries of the object. The supervisor may be able to provide input to the state sensing device asking for a different object, or different boundaries of an object to be selected. The supervisor may be able to accept the object and/or boundaries by some input to the state sensing device, such as a click button, touch screen entry, voice command, etc. Note that the accurate distance from the state sensing device provided by the range sensor 320 (FIG. 3) may be useful in differentiating which object in an environment a supervisor is designating as a target object, as well as confirming a state of the object later.

Furthermore, objects selected by a supervisor during a training process may be edited later. For instance, more or fewer target objects may be edited, boundaries may be adjusted using stored image data, objects may be annotated (e.g., “sink,” “bed”), etc. Editing functionality may be provided via the state sensing device, or the state sensing device may provide training process information to another device, such as a computing device 208 (FIG. 2) and/or a network device 204. In some examples, application module 258 of computing device 208 and/or application module 232 of network device 204 may assist with editing functionality related to target objects.

In some examples, a training process could include mapping a canonical environment. For instance, in a hotel in which most of the rooms appear relatively similar, such as having similar dimensions and furniture, a template may be made from one representative room. A supervisor may perform a training process using the state sensing device in the representative room, and expect that the training process information will be applicable to the other hotel rooms of the hotel. Similarly, if rooms are similar, the state sensing device may be able to help determine that a chair is moved to an unexpectedly location in a room.

Additionally, in some implementations a state sensing device may be relatively “aware” of its geospatial positioning. For instance, the state sensing device may be aware of which hotel and/or room it is in, in the context of a cleaning program. The state sensing device may be aware that it has been in a particular room previously. The state sensing device may be aware that there is a checklist for a particular room, such as a checklist that has been used previously for that room. The state sensing device may then be able to automatically bring up the checklist for display to the employee 902 and/or direct the employee to objects that need to be disinfected. In another example, an anchor point may be established and/or used with the state sensing device. For instance, a particular location may be designated as an anchor point. The state sensing device may be aware when it arrives at an anchor point and/or may automatically follow certain instructions when it arrives at an anchor point. Stated another way, the state sensing device may determine a location of the device in an environment and can update the location as the device is moved about the environment.

Training process information may be used to create a variety of management tools for ensuring task completion by employees, such as cleaning crews. For instance, target objects in an environment may be listed in a program and automatically checked off as an employee completes tasks with the state sensing device. For instance, as the employee moves about environment 900, the program may automatically check off the bed, the bathroom sink, etc., as the employee completes disinfection of each object. A supervisor may manually review the list, or may automatically receive an indication that the job is done, such as via a computing device 208 (FIG. 2). In other instances, a state sensing device may be able to provide an efficiency score for an employee, such as efficiency in use of materials (e.g., using an appropriate amount of disinfectant spray), efficiency in motion or use of time (e.g., how many passes over a room with a vacuum an employee uses to vacuum an entire room adequately), etc. In some cases, developer module 234 of network device(s) 204 (FIG. 2) may provide functionality relating to developing management tools for supervisors or companies. For instance, a hotel chain company or an airline company may be interested in developing tools or programs that are specific to their standards and/or associated environments. As such, a representative from a hotel chain company or an airline company may be able to access developer module 234. Developer module 234 may provide an application program interface (API) for the representative to customize aspects of the state determination functionality. For instance, the representative may customize classification parameters and/or thresholds, certification sensitivity (e.g., a confidence level for disinfection), various output formats and/or content, etc.

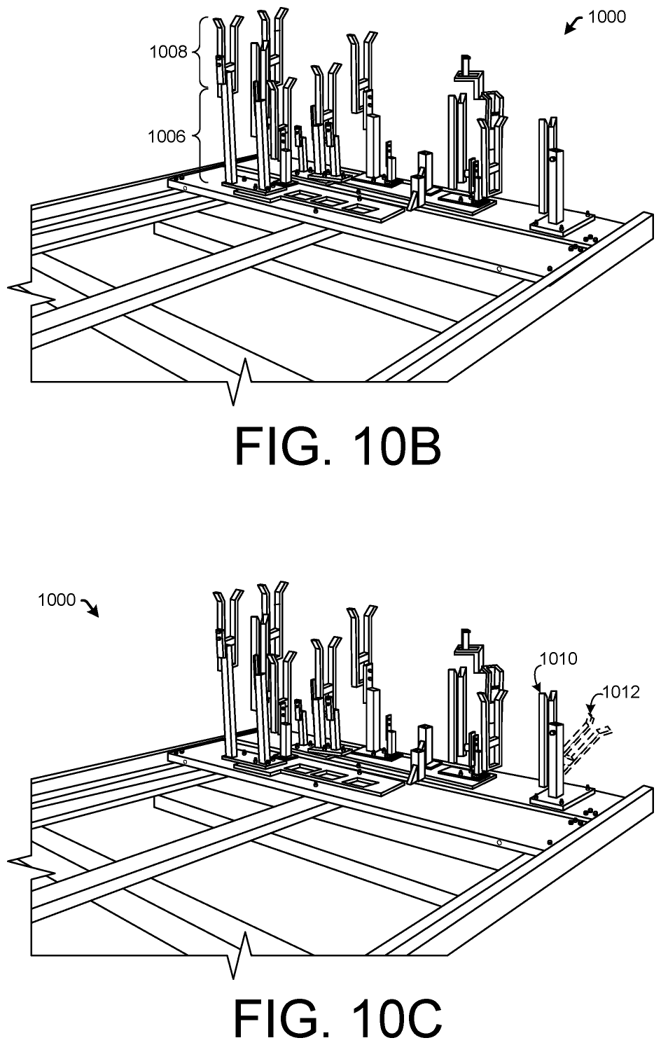

FIGS. 10A-10C include partial cutaway perspective views of an example environment 1000. In this example, environment 1000 is an assembly line in an automobile manufacturing facility. FIG. 10A illustrates an example scenario in which a pallet 1002 supports a car part 1004. For instance, pallet 1002 may support the car part 1004 in a particular position as it moves along the assembly line. FIG. 10B shows pallet 1002 without the car part 1004, so that detail of the supporting structures may be seen. As shown in FIG. 10B, pallet 1002 may include arms 1006. Arms 1006 may include contactors 1008 (only one arm 1006 and one contactor 1008 is designated with specificity for ease of understanding). A contactor 1008 may be positioned at a distal end of an arm 1006 (e.g., away from pallet 1002) for the purpose of receiving, or “contacting,” the car part 1004. Although not visible in FIGS. 10A-10C, environment 1000 may include a state sensing device mounted in the facility, similar to state sensing device 700 in FIG. 7, or may include a state sensing device mounted on a robot arm, similar to state sensing device 800 in FIG. 8. The state sensing device may be positioned and/or aimed within the facility to have a view of equipment, such as pallet 1002, arms 1006, contactors 1008, car part 1004, and/or other objects.

FIG. 10C depicts an instance where contactor 1010 has been deformed, indicated in dashed outline at 1012. More specifically, in this scenario contactor 1010 has been deformed from the shape and/or position shown in solid lines in FIGS. 10B and 10C to the deformed outline at 1012. As used herein, deformation may include a change in shape of an object and/or a variety of other states of the object related to shape and/or position, such as a loss of elasticity or a loss of spring action which prevents an object from rebounding and/or resetting to an expected shape, for instance. In some examples, a state sensing device may collect information regarding a state of pallet 1002, arms 1006, contactors 1008, car part 1004, and/or other objects located in an environment similar to example environment 1000. The state sensing device may collect information regarding precise positioning of objects, wear and tear on objects, maintenance and repair of objects, predictive maintenance over time, etc. For example, the deformation of contactor 1010 may correspond to damage that occurred to the contactor 1010 when a previous car part was unloaded, bending the contactor 1010 to the deformed shape 1012. In the instance where contactor 1010 is deformed, contactor 1010 may not be able to properly receive (e.g., seat, support) car part 1004. A state sensing device may be able to recognize the deformation of contactor 1010. The state sensing device may then alert a supervisor of the assembly line that a problem exists.

Similarly, the deformation 1012 may have occurred over time, as a progressive degradation in the integrity of the contactor 1010 over the course of supporting many car parts. A state sensing device may consider a threshold amount of deformation before classifying the contactor 1010 as deformed, for instance. Further, a state sensing device may track an amount of time that has passed since a maintenance process was performed on equipment, such as pallet 1002, arms 1006, and contactors 1008. For instance, a state sensing device may determine that three months has passed since the last maintenance work, and that pallet 1002 is due for a maintenance inspection. Further, in addition to sensing issues itself, the state sensing device may communicate with one or more other humans, robots, state sensing devices, and/or other devices to combine information for assessing a condition of an object(s) and/or general conditions in an environment. Therefore, a state sensing device may participate in “co-botics.”

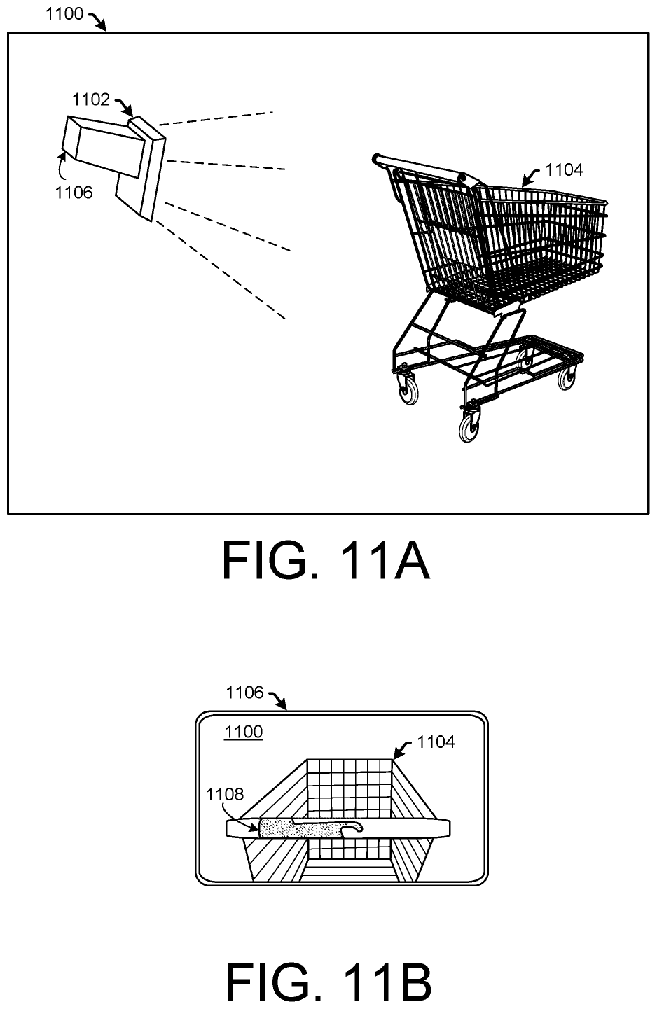

FIGS. 11A and 11B include perspective views of an example environment 1100. Environment 1100 may include a state sensing device 1102 and an object 1104 (e.g., a shopping cart). State sensing device 1102 may include a display 1106. Note that FIG. 11B represents the view shown on the display 1106. Example environment 1100 shown in FIG. 11A corresponds to an example scenario in which state sensing device 1102 includes alternative functionality than the example state sensing devices described above. For instance, example state sensing device 1102 may include disinfection functionality in the form of emitted UV light. In general, some componentry described below may be similar to componentry described above. In cases of similar componentry, descriptions will not be repeated for sake of brevity. As used herein, “sanitized” may refer to “disinfected.”

State sensing device 1102 may be configured to sanitize surfaces of furniture, products, goods, or anything with a visible surface. In preferred embodiments, augmented-Reality (AR) enabled hardware may be combined with a high-power Ultraviolet (UV)-based sanitization light system to enable user-aware sanitization of rooms, spaces, objects, and the like. The AR software may be joined to a dose calculation that may be based on the distance and angle from and power of, a known UV sanitizing element. Preferably, visual feedback is provided to the user, such as in the form of a color-coded AR overlay to the surroundings. In such an embodiment, a color change may signify a sufficient dose to achieve a desired level of sanitization. In further enhancements, the system may produce a certification for the particular level of sanitization achieved.

Generally described, the example shown in FIGS. 11A and 11B is directed at a mechanism and technique to sanitize a surface of an object 904. Embodiments of the disclosure include a state sensing device 1102 that irradiates ultraviolet light at the surface of object 1104. State sensing device 1102 may include a sensor, such as a camera, to image object 1104 that is under irradiation. State sensing device 1102 may be able to determine a level of sanitization of object 1104, such as through direct evaluation or estimation based on the amount of treatment applied to the object. State sensing device 1102 may then display object 1104 with an augmented overlay to show a level of sanitization that has been achieved. Preferred embodiments will now be described.

State sensing device 1102 may be incorporated in or include a mobile device (such as a mobile phone), desktop computer, laptop computer, email/messaging device, tablet computer, or similar device that may be configured to perform the functions described herein. Generally, state sensing device 1102 may be implemented with any type of computing device or any device that is configured to process data in accordance with methods and functions described herein.

In certain embodiments, state sensing device 1102 may include an interface, a wireless communication component, a cellular radio communication component, a global positioning system (GPS) receiver, sensor(s), data storage, and processor(s). State sensing device 1102 may also include hardware to enable communication between the state sensing device 1102 and other computing devices, such as network devices 204 (FIG. 2). The hardware may include transmitters, receivers, and antennas, for example.

The interface may be configured to allow state sensing device 1102 to communicate with other computing devices, such as network devices 204 (FIG. 2). Thus, the interface may be configured to receive input data from one or more computing devices, and may also be configured to send output data to the one or more computing devices. The interface may be configured to function according to a wired or wireless communication protocol. In some examples, the interface may include buttons, a keyboard, a touchscreen, speaker(s), microphone(s), and/or any other elements for receiving inputs, as well as one or more displays, and/or any other elements for communicating outputs.

The wireless communication component may be a communication interface that is configured to facilitate wireless data communication for state sensing device 1102 according to one or more wireless communication standards. For example, the wireless communication component may include a Wi-Fi communication component that is configured to facilitate wireless data communication according to one or more IEEE 802.11 standards. As another example, the wireless communication component may include a Bluetooth communication component that is configured to facilitate wireless data communication according to one or more Bluetooth standards. Other examples are also possible.

The cellular radio communication component may be a communication interface that is configured to facilitate wireless communication (voice and/or data) with a cellular wireless base station to provide mobile connectivity to a network. The cellular radio communication component may be configured to connect to a base station of a cell in which state sensing device 1102 is located, for example.

The GPS receiver may be configured to estimate a location of state sensing device 1102 by precisely timing signals received from Global Positioning System (GPS) satellites.

The sensor(s) may include one or more sensors, or may represent one or more sensors included within or coupled to state sensing device 1102. Example sensors include an accelerometer, gyroscope, pedometer, LIDAR or other optical sensors, microphone, camera(s), infrared flash, barometer, magnetometer, Wi-Fi, near field communication (NFC), Bluetooth, projector, depth sensor, temperature sensor, or other location and/or context-aware sensors.

The data storage (memory) may store program logic that can be accessed and executed by the processor(s). The data storage may also store data collected by the sensor(s), or data collected by any of the wireless communication component, the cellular radio communication component, and the GPS receiver.

The processor(s) may be configured to receive data collected by any of sensor(s) and perform any number of functions based on the data. As an example, the processor(s) may be configured to determine one or more geographical location estimates of state sensing device 1102 using one or more location-determination components, such as the wireless communication component, the cellular radio communication component, or the GPS receiver. The processor(s) may use a location-determination algorithm to determine a location of state sensing device 1102 based on a presence and/or location of one or more known wireless access points within a wireless range of state sensing device 1102.

The communication link may be a wired or wireless connection. For example, the communication link may be a wired serial bus such as a universal serial bus or a parallel bus, or a wireless connection using, e.g., shortrange wireless radio technology, or communication protocols described in IEEE 802.11 (including any IEEE 802.11 revisions), among other possibilities.

State sensing device 1102 may include more or fewer components. Further, example methods described herein may be performed individually by components of state sensing device 1102, or in combination by one or all of the components of state sensing device 1102.

In preferred embodiments, state sensing device 1102 is further coupled to an ultraviolet (“UV”) light source. In such embodiments, state sensing device 1102 may be further configured to control the operation of the UV light source. For example, state sensing device 1102 may control an amount of power irradiated by the light source and a duration of operation. State sensing device 1102 is, accordingly, aware of how much sanitization treatment is applied by the UV light source and the area over which that treatment was applied. In this way, state sensing device 1102 is able to either directly measure or estimate an amount of sanitization that any particular object has received.

Several manufacturers of tablet/phone technology include AR toolsets for use by developers (e.g. Apple AR Kit). These products pair enhanced measurement of the area surrounding the mobile device (generally driven by LIDAR or optical sensors) with AI-based space estimation methods to produce an enhanced, 3D awareness of the environment around the mobile device. These allow for the device to encode a 3D object model for the room in which it is placed with minimal effort on the part of the user, and allows for “enhancements” to be rendered over this room model on a display, such as a user interface. These enhancements can include things like freestanding objects projected into the space, but also varying color and other qualities of existing surfaces.

Certain sanitization techniques may be implemented in various embodiments. For example, ultraviolet germicidal irradiation (UVGI) is a disinfection method that typically uses short-wavelength ultraviolet (UV-C) light to kill or inactivate microorganisms by destroying nucleic acids and disrupting their DNA, leaving them unable to perform vital cellular functions. UV-based sanitization devices are known and available. Generally, these are either high power fluorescent mercury-vapor lamps, or LED-based solutions, in either case generating photons in the UV-C spectrum (around 200 to 280 nm). This spectrum has been studied extensively and shown to accomplish sterilization and sanitization of pathogens given sufficient dose. There are several solutions capable of accomplishing significant disinfection in several seconds from a few feet away, and dosage can be easily calculated given a known emitter (e.g., a bulb) and the time and distance spent in proximity to a surface.

In preferred embodiments of the disclosure, the two technologies just described are combined into a single handheld device (e.g., state sensing device 1102), with the emitter positioned in a way such that the visual field of the device displays areas/surfaces affected by the emitter. The AR kit/facility of the tablet may be used to recolor surfaces to indicate untreated status (e.g., surfaces that have not accumulated any UV-C dose or sufficient UV-C dose to achieve approval as sanitized).

The device may include an actuator (e.g. a trigger system) to turn on the emitter and inform the tablet/computer system when the emitter is on and producing UV light. The device performs a calculation based on the distance between the emitter and each known surface (as determined by the sensors) to determine an accumulated dose on each surface. The device employs the AR kit to recolor surfaces to indicate total estimated pathogen reduction, and may be scaled to a desired level of total reduction. This allows the user to use the sanitizing device after the fashion of a power washer or airbrush, using color change in real-time as an indication of level of dose accumulated over the space or object.

The accumulated knowledge of surfaces in an area or object may be used to generate certificates of sanitization, in which surfaces (or an average of surfaces) can be shown to have accumulated enough dose to achieve desired sanitization.

Referring again to FIGS. 11A and 11B, a conceptual diagram generally illustrates a sample display of a surface showing various levels of sanitization achieved on the surface of object 1104. As shown in FIG. 11A, an environment 1100 includes an object 1104. In this example, the object 1104 is a shopping cart but may be any other object, such as furniture in an office or shelves with products for sale. A sanitizing device 1102 is configured as described above and is situated so as to irradiate the object 1104. The sanitizing device 1102 further includes a display 1106. The sanitizing device 1102 is configured to monitor the environment 1100 and the effects of irradiating the environment with a sanitizing treatment, such as UV-C light.

The sanitizing device 1102 employs AR technology to render on the display 1106 an image of the environment 1100, including the object 1104 under treatment (see FIG. 11B). On top of (e.g., overlain) the image of the environment 1100 is rendered an indication whether, and perhaps to what extent, pathogen reduction has occurred. For example, in the case where the sanitization device 1102 has not yet adequately sanitized a portion (e.g., handle 1108) of the object 1104, that portion may be rendered in a different color or with some other indication of a lack of sufficient sanitization. In one specific implementation, the as-yet unsanitized portion of the object may be rendered with a shaded overlay to indicate that it is not yet sanitized.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Additional functionality of the operations and components described above with reference to FIGS. 1-11B is discussed with reference to various flow diagrams and examples shown throughout the disclosure.

FIGS. 1 and 12-14 show flow diagrams that illustrative various example processes. The processes are illustrated as a collection of blocks in a logical flow graph, which represent a sequence of operations that can be implemented in hardware, software, or a combination thereof. In some instances, the collection of blocks is organized under respective entities that may perform the various operations described in the blocks. In the context of software, the blocks represent computer-executable instructions stored on one or more computer storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the processes.

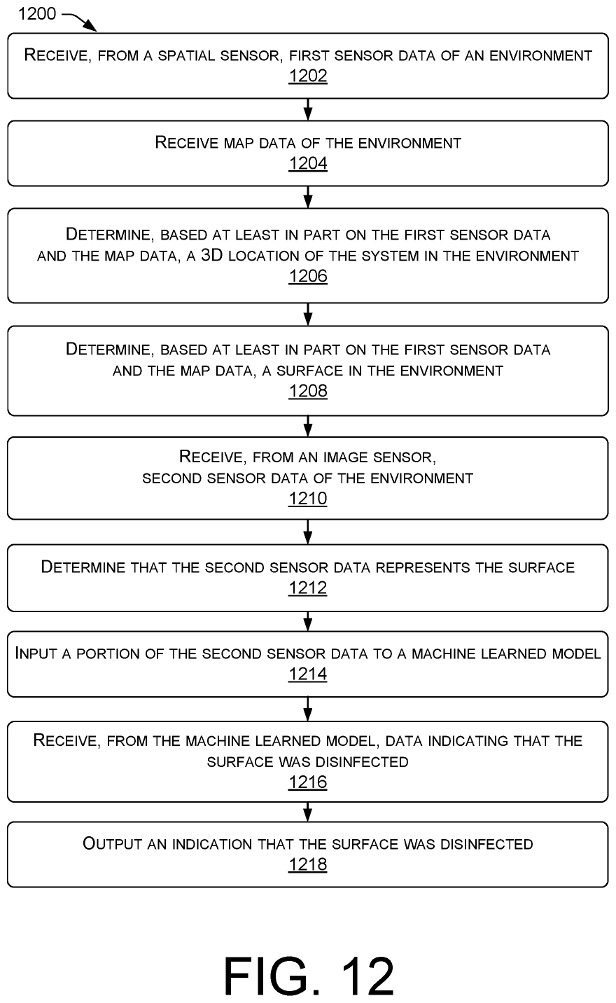

FIG. 12 is a flow diagram of an illustrative process 1200 for determining a state of a surface in an environment. In general, process 1200 is a process for determining whether the surface was disinfected. Process 1200 may be performed by a state sensing device, such as state sensing device 112, 202, 300, 500, 600, 700, 800, or 1102. Process 1200 may be performed in the environments introduced in the examples described above, and/or in other similar and/or different environments.

At 1202, the operation can include receiving, from a spatial sensor, first sensor data of an environment. In some examples, the spatial sensor may comprise at least one of a structured light camera and a time-of-flight camera.

At 1204, the operation can include receiving map data of the environment.

At 1206, the operation can include determining, based at least in part on the first sensor data and the map data, a three-dimensional (3D) location of the system in the environment.

At 1208, the operation can include determining, based at least in part on the first sensor data and the map data, a surface in the environment. For example, determining a surface may include segmenting the environment into one or more segments, wherein the determining the surface is based at least in part on the one or more segments. In some implementations, the segmenting may be based at least in part on a connected component algorithm.

At 1210, the operation can include receiving, from the image sensor, second sensor data of the environment.

At 1212, the operation can include determining that the second sensor data represents the surface.

At 1214, the operation can include inputting a portion of the second sensor data to a machine learned model.

At 1216, the operation can include receiving, from the machine learned model, data indicating that the surface was disinfected.

At 1218, the operation can include outputting an indication that the surface was disinfected. In some examples, the operation can include causing the indication that the surface was disinfected to be presented on a display.

In some examples, the operation may further include causing a UV light source to irradiate the surface. In this example, the operation can include providing an indication that the UV light source is irradiating (or has irradiated) the surface to the machine learned model. In some implementations, data indicating that the surface was disinfected may be based at least in part on the indication that the UV light source is irradiating (or has irradiated) the surface.

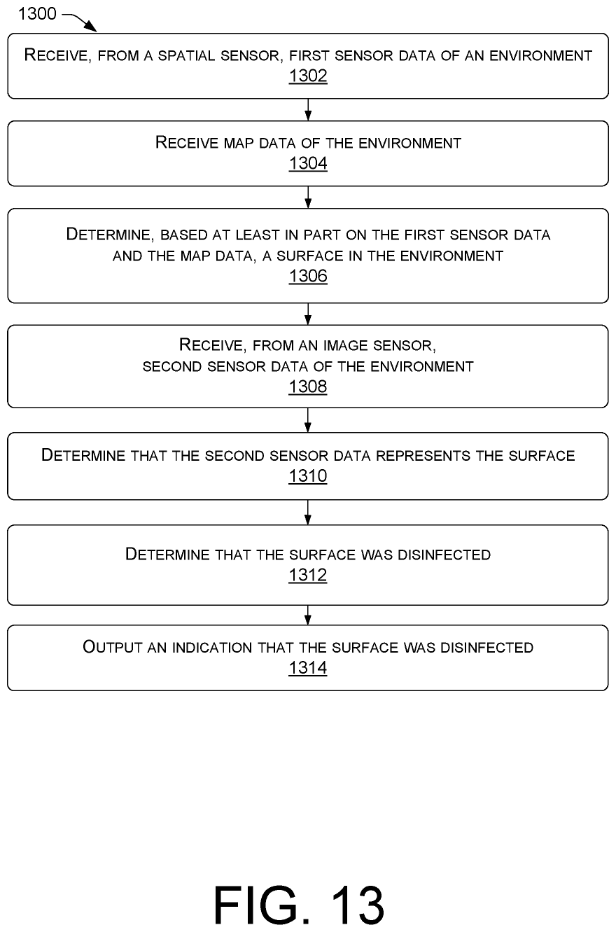

FIG. 13 is a flow diagram of an illustrative process 1300 for determining a state of a surface in an environment. In general, process 1300 is a process for determining whether the surface was disinfected. Process 1300 may be performed by a state sensing device, such as state sensing device 112, 202, 300, 500, 600, 700, 800, or 1102. Process 1300 may be performed in the environments introduced in the examples described above, and/or in other similar and/or different environments.

At 1302, the operation can include receiving, from a spatial sensor, first sensor data of an environment.

At 1304, the operation can include receiving map data of the environment.

At 1306, the operation can include determining, based at least in part on the first sensor data and the map data, a surface in the environment. In some examples, the operation can include creating a pixel depth map of the environment. The operation may further include transforming the pixel depth map to a tensor map (e.g., a tensor field). In some examples, the determination of the surface may be based at least in part on the tensor map. The tensor map may include a vector in a direction of greatest change in depth, for instance. Furthermore, the operation may include receiving information indicating that the surface is a relevant surface of interest in the environment. The determining the surface is based at least in part on the surface being the relevant surface of interest.

At 1308, the operation can include receiving, from an image sensor, second sensor data of the environment.

At 1310, the operation can include determining that the second sensor data represents the surface.

At 1312, the operation can include determining that the surface was disinfected. In some examples, the operation may include inputting a portion of the second sensor data to a machine learned model. The determining that the surface was disinfected may be based at least in part on the machine learned model using the portion of the second sensor data, for instance. The machine learned model may comprise a support vector machine (SVM) algorithm, in some instances.

In some instances, determining that the surface was disinfected may include fusing at least a portion of the first sensor data and at least a portion of the second sensor data to create a combined view of the environment. The determination that the surface was disinfected may then be based at least in part on the combined view of the environment.

At 1314, the operation can include outputting an indication that the surface was disinfected. In some examples, determining that the surface was disinfected may be made on a per-pixel basis relative to the second sensor data. The indication that the surface was disinfected may reference the per-pixel basis of disinfection. For instance, the indication that the surface was disinfected may include a specification related to an amount of the surface that was disinfected. In other examples, a visual display may be generated based at least in part on the combined view of the environment suggested above. The outputting the indication that the surface was disinfected may then comprise outputting the visual display. For instance, the operation may include causing the visual display to be presented on a display device. In other examples, outputting the indication that the surface was disinfected may comprise producing a haptic output.

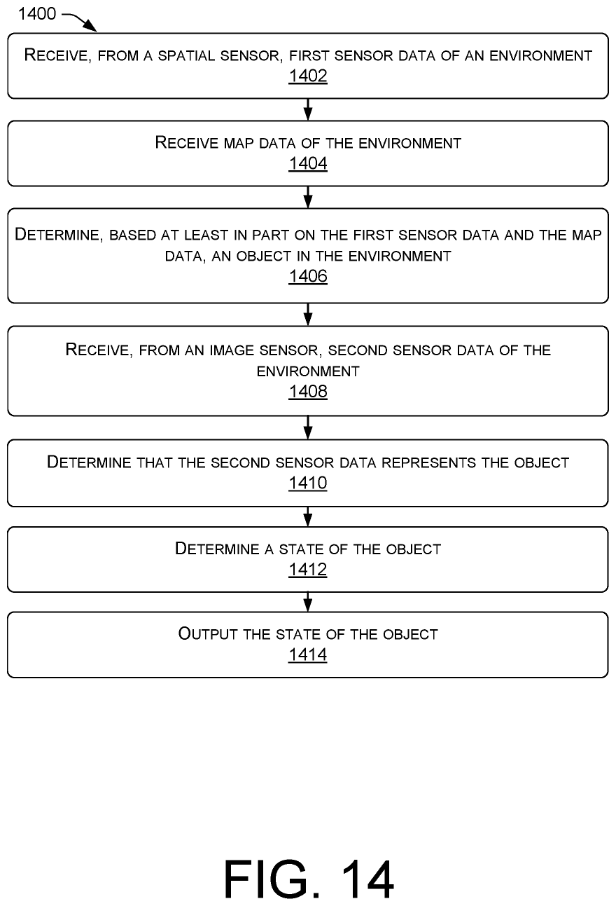

FIG. 14 is a flow diagram of an illustrative process 1400 for determining a state of an object in an environment. Process 1400 may be performed by a state sensing device, such as state sensing device 112, 202, 300, 500, 600, 700, 800, or 1102. Process 1400 may be performed in the environments introduced in the examples described above, and/or in other similar and/or different environments.

At 1402, the operation can include receiving, from a spatial sensor, first sensor data of an environment.

At 1404, the operation can include receiving map data of the environment;

At 1406, the operation can include determining, based at least in part on the first sensor data and the map data, an object in the environment. In some examples, the object may be a surface in the environment.

At 1408, the operation can include receiving, from an image sensor, second sensor data of the environment.

At 1410, the operation can include determining that the second sensor data represents the object.

At 1412, the operation can include determining a state of the object. In some examples, the state may refer to a disinfection status of the object. In other examples, the object may be an assembly product, and the state of the object may indicate whether a component has been assembled onto the assembly product.

At 1414, the operation can include outputting the state of the object.

Thus, a state sensing device can be utilized in conjunction with one or more computing devices, accessory devices, and/or network devices to provide information and/or a certification regarding a state of an object in an environment, such as whether an object has likely been disinfected.

### CONCLUSION

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

What is claimed is:

1. A system comprising:
a spatial sensor;
an image sensor;
one or more processors; and
one or more non-transitory computer-readable media storing computer-executable instructions that, when executed, cause the system to perform operations comprising:
receiving, from the spatial sensor, first sensor data of an environment;
receiving map data of the environment;
determining, based at least in part on the first sensor data and the map data, a three-dimensional (3D) location of the system in the environment;
determining, based at least in part on the first sensor data and the map data, a surface in the environment;
receiving, from the image sensor, second sensor data of the environment;
determining that the second sensor data represents the surface;
inputting a portion of the second sensor data to a machine learned model;
receiving, from the machine learned model, data indicating that the surface was disinfected; and
outputting an indication that the surface was disinfected.

2. The system of claim 1, the operations further comprising:
segmenting the environment into one or more segments, wherein the determining the surface is based at least in part on the one or more segments.

3. The system of claim 2, wherein the segmenting is based at least in part on a connected component algorithm.

4. The system of claim 2, wherein the spatial sensor comprises at least one of a structured light camera or a time-of-flight camera.

5. The system of claim 1, further comprising:
an ultraviolet (UV) light source,
wherein the operations further comprise:
causing the UV light source to irradiate the surface; and
providing an indication that the UV light source is irradiating the surface to the machine learned model,
wherein the data indicating that the surface was disinfected is based at least in part on the indication.

6. A method comprising:
receiving, from a spatial sensor, first sensor data of an environment;
receiving map data of the environment;
determining, based at least in part on the first sensor data and the map data, a surface in the environment;
receiving, from an image sensor, second sensor data of the environment;
determining that the second sensor data represents the surface;
determining that the surface was disinfected; and
outputting an indication that the surface was disinfected.

7. The method of claim 6, further comprising:
creating a pixel depth map of the environment; and
transforming the pixel depth map to a tensor map;
wherein the determining the surface is based at least in part on the tensor map.

8. The method of claim 7, wherein the tensor map includes a vector indicative of a direction of a change in depth.

9. The method of claim 6, further comprising:
receiving information indicating that the surface is a relevant surface of interest in the environment, wherein the determining the surface is based at least in part on the surface being the relevant surface of interest.

10. The method of claim 6, further comprising:
inputting a portion of the second sensor data to a machine learned model,
wherein the determining that the surface was disinfected is based at least in part on the machine learned model using the portion of the second sensor data.

11. The method of claim 10, wherein the machine learned model comprises a support vector machine (SVM) algorithm.

12. The method of claim 6, wherein the determining that the surface was disinfected is made on a per-pixel basis relative to the second sensor data.

13. The method of claim 12, wherein the indication that the surface was disinfected references the per-pixel basis, indicating an amount of the surface that was disinfected.

14. The method of claim 6, further comprising:
fusing at least a portion of the first sensor data and at least a portion of the second sensor data to create a combined view of the environment,
wherein the determining that the surface was disinfected is based at least in part on the combined view of the environment.

15. The method of claim 14, further comprising:
generating a visual display based at least in part on the combined view of the environment, and
wherein the outputting the indication that the surface was disinfected comprises outputting the visual display.

16. The method of claim 15, further comprising:
causing the visual display to be presented on a display device.

17. The method of claim 6, wherein the outputting the indication that the surface was disinfected comprises producing a haptic output.

18. One or more non-transitory computer-readable media storing computer-executable instructions that, when executed, cause one or more processors to perform operations comprising:
receiving, from a spatial sensor, first sensor data of an environment;
receiving map data of the environment;
determining, based at least in part on the first sensor data and the map data, an object in the environment;
receiving, from an image sensor, second sensor data of the environment;
determining that the second sensor data represents the object;
determining a state of the object; and
outputting the state of the object.

19. The one or more non-transitory computer-readable media of claim 18, wherein the object is a surface in the environment, and the state of the object refers to a disinfection status of the object.

20. The one or more non-transitory computer-readable media of claim 18, wherein the object is an assembly product, and the state of the object indicates whether a component has been assembled onto the assembly product.
